(19) United States
(12) Patent Application Publication
(10) Pub. No.: US 2026/0060606 A1
(43) Pub. Date: Mar. 5, 2026

(54) APPARATUSES FOR FETAL HEALTH MONITORING

(71) Applicant: MISSMEDICAL VENTURES 1 LLC, Fairhope, AL (US)

(72) Inventors: Megan E Missanelli, Fairhope, AL (US); Kirk S. Honour, EXCELSIOR, MN (US)

(73) Assignee: MISSMEDICAL VENTURES 1 LLC, Fairhope, AL (US)

(21) Appl. No.: 19/385,185

(22) Filed: Nov. 11, 2025

Related U.S. Application Data
(60) Provisional application No. 63/774,155, filed on Mar. 19, 2025.

(51) Int. Cl. A61B 5/00; A61B 5/0205; A61B 5/1455; A61B 5/287; A61B 5/288

(52) U.S. Cl. CPC A61B 5/4362; A61B 5/02055; A61B 5/14552; A61B 5/287; A61B 5/288; A61B 2560/0214; A61B 2560/063; A61B 2562/16; A61B 2562/166; A61B 2562/185

(57) ABSTRACT

The present disclosure provides an apparatus for fetal health monitoring. Further, the apparatus includes a body, an electrode extending between a first electrode end and a second electrode end, the second electrode end configured to be inserted into a body part of a fetus for securing the apparatus to the body parts based on an action receivable on the body. Further, the electrode is configured for detecting an electrical signal associated with a first physiological parameter of the fetus. Further, the apparatus includes a sensor configured for detecting a second physiological parameter associated with the fetus. Further, the apparatus includes a processing device configured for generating a first physiological data and a second physiological data. Further, the apparatus includes a communication device configured for transmitting the first physiological data and the second physiological data to an external device.

### REFERENCE TO RELATED APPLICATIONS

This application claims the benefit of U.S. Provisional Patent Application No. 63/774,155, titled “DEVICE FOR FACILITATING A FETUS HEALTH MONITORING”, filed on Mar. 19, 2025, which is incorporated by reference herein in its entirety. Further, the current application is also related to co-owned U.S. patent application Ser. No. 18/342,143, titled “System for monitoring fetal status during childbirth”, filed on Jun. 27, 2023, the entirety of which is incorporated herein by reference.

### FIELD OF DISCLOSURE

The present disclosure generally relates to the field of medical and laboratory equipment. More specifically, the present disclosure relates to apparatuses for fetal health monitoring.

### BACKGROUND

The field of intrapartum fetal monitoring is of critical importance to maternal-fetal medicine and neonatal care. Monitoring fetal health during labor is essential to safeguard against hypoxia, acidosis, and other complications that may lead to severe neonatal morbidity or mortality. The ability to obtain accurate, reliable, and timely physiological data from the fetus allows clinicians to make informed decisions that may reduce the risks of operative deliveries, improve outcomes for both mother and child, and support the delivery process safely and effectively.

A desirable objective in the disclosed field is to achieve continuous, multi-parameter fetal monitoring that provides accurate physiological information, enhances diagnostic precision, minimizes false positives, and reduces unnecessary interventions. Such an objective would support timely and appropriate responses to fetal distress, enable more precise correlation of fetal condition with maternal factors, and ultimately improve perinatal outcomes.

However, existing approaches to fetal monitoring often present significant limitations in achieving the disclosed objective. Current techniques may provide incomplete or indirect indicators of fetal well-being, leading to challenges in detecting actual hypoxic or acidotic conditions. In many cases, monitoring systems are susceptible to noise, signal loss, or confounding maternal physiological factors, which may diminish the accuracy of the monitoring systems. Conventional approaches may also lack the ability to directly measure critical physiological parameters and may require invasive, time-consuming, or disruptive procedures that increase risks to both the mother and fetus. The limitations may contribute to a high incidence of false alarms, variability in interpretation, and a corresponding increase in unnecessary surgical interventions. In addition, disparities in access to skilled obstetric care may compound the limitations, as many systems lack the connectivity and remote monitoring capabilities necessary to extend specialized oversight to under-resourced settings.

Therefore, there is a need for improved apparatuses for fetal health monitoring that may overcome one or more of the preceding problems.

### SUMMARY OF DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure provides an apparatus for fetal health monitoring. Further, the apparatus may include a body extending between a first body end and a second body end.

Further, the body includes a body cavity. Further, the first body end defines a body opening leading into the body cavity. Further, the body may be configured to be inserted into one or more user body cavities of one or more users. Further, the apparatus may include an electrode extending between a first electrode end and a second electrode end. Further, the electrode may be helically extending from the body. Further, the first electrode end may be attached to the body, and the second electrode end may be free. Further, the second electrode end may be configured to be inserted into one or more body parts of a fetus for securing the apparatus to the one or more body parts based on one or more actions receivable on the body. Further, the electrode may be configured for detecting one or more electrical signals associated with the one or more body parts of the fetus based on the inserting of the second electrode end into the one or more body parts of the fetus. Further, the one or more electrical signals may be associated with one or more first physiological parameters of the fetus. Further, the apparatus may include one or more sensors disposed in the body cavity. Further, the one or more sensors may be configured for detecting one or more second physiological parameters associated with the fetus. Further, the apparatus may include a processing device disposed within the body. Further, the processing device may be communicatively coupled with the electrode and the one or more sensors. Further, the processing device may be configured for generating one or more first physiological data based on the detecting of the one or more electrical signals. Further, the processing device may be configured for generating one or more second physiological data based on the detecting of the one or more second physiological parameters. Further, the apparatus may include a communication device disposed within the body. Further, the communication device may be communicatively coupled with the processing device. Further, the communication device may be configured for transmitting the one or more first physiological data and the one or more second physiological data to one or more external devices.

The present disclosure provides an apparatus for fetal health monitoring. Further, the apparatus may include a body extending between a first body end and a second body end.

Further, the body includes a body cavity. Further, the first body end defines a body opening leading into the body cavity. Further, the body may be configured to be inserted into one or more user body cavities of one or more users. Further, the apparatus may include an electrode extending between a first electrode end and a second electrode end. Further, the electrode may be helically extending from the body. Further, the first electrode end may be attached to the body, and the second electrode end may be free. Further, the second electrode end may be configured to be inserted into one or more body parts of a fetus for securing the apparatus to the one or more body parts based on one or more actions receivable on the body. Further, the electrode may be configured for detecting one or more electrical signals associated with the one or more body parts of the fetus based on the inserting of the second electrode end into the one or more body parts of the fetus. Further, the one or more electrical signals may be associated with one or more first physiological parameters of the fetus. Further, the electrode further includes an electrode cavity extending from the second electrode end. Further, the second electrode end defines an electrode opening leading into the electrode cavity. Further, the electrode further includes a sample collecting mechanism coupled with the electrode cavity. Further, the sample collecting mechanism may be configured to be activated. Further, the sample collecting mechanism may be configured for collecting one or more samples associated with the fetus based on the inserting of the body into the one or more user body cavities of the one or more users after the activating of the sample collecting mechanism. Further, the electrode may be configured for storing the one or more samples in the electrode cavity based on the collecting of the one or more samples. Further, the apparatus may include one or more sensors disposed in the body cavity. Further, the one or more sensors may be configured for detecting one or more second physiological parameters associated with the fetus. Further, the apparatus may include a processing device disposed within the body. Further, the processing device may be communicatively coupled with the electrode and the one or more sensors. Further, the processing device may be configured for generating one or more first physiological data based on the detecting of the one or more electrical signals. Further, the processing device may be configured for generating one or more second physiological data based on the detecting of the one or more second physiological parameters. Further, the apparatus may include a communication device disposed within the body. Further, the communication device may be communicatively coupled with the processing device. Further, the communication device may be configured for transmitting the one or more first physiological data and the one or more second physiological data to one or more external devices.

The present disclosure provides an apparatus for fetal health monitoring. Further, the apparatus may include a body extending between a first body end and a second body end. Further, the body includes a body cavity. Further, the first body end defines a body opening leading into the body cavity. Further, the body may be configured to be inserted into one or more user body cavities of one or more users. Further, the apparatus may include an electrode extending between a first electrode end and a second electrode end. Further, the electrode may be helically extending from the body. Further, the first electrode end may be attached to the body, and the second electrode end may be free. Further, the second electrode end may be configured to be inserted into one or more body parts of a fetus for securing the apparatus to the one or more body parts based on one or more actions receivable on the body. Further, the electrode may be configured for detecting one or more electrical signals associated with the one or more body parts of the fetus based on the inserting of the second electrode end into the one or more body parts of the fetus. Further, the one or more electrical signals may be associated with one or more first physiological parameters of the fetus. Further, the apparatus may include one or more sensors disposed in the body cavity. Further, the one or more sensors may be configured for detecting one or more second physiological parameters associated with the fetus. Further, the apparatus may include a processing device disposed within the body. Further, the processing device may be communicatively coupled with the electrode and the one or more sensors. Further, the processing device may be configured for generating one or more first physiological data based on the detecting of the one or more electrical signals. Further, the processing device may be configured for generating one or more second physiological data based on the detecting of the one or more second physiological parameters. Further, the apparatus may include a communication device disposed within the body. Further, the communication device may be communicatively coupled with the processing device. Further, the communication device may be configured for transmitting the one or more first physiological data and the one or more second physiological data to one or more external devices. Further, the apparatus may include a delivery tube extending between a first tube end and a second tube end. Further, the delivery tube includes a tube cavity extending between the first tube end and the second tube end. Further, the body may be disposed in the tube cavity at the first tube end. Further, the first tube end may be inserted in the one or more user body cavities. Further, the first tube end may be configured to be positioned in one or more positions in the one or more user body cavities in relation to the fetus after the inserting of the first tube end. Further, the inserting of the second electrode end into the one or more body parts of the fetus may be based on the positioning of the first tube end in the one or more positions. Further, the one or more actions receivable on the body may be applied using the delivery tube.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 illustrates an apparatus 100 for fetal health monitoring, in accordance with some embodiments.

FIG. 2 illustrates a cross-sectional view of the apparatus 100 for fetal health monitoring, in accordance with some embodiments.

FIG. 3 illustrates an electrode 112 of the apparatus 100 for fetal health monitoring, in accordance with some embodiments.

FIG. 4 illustrates the electrode 112 of the apparatus 100 for fetal health monitoring, in accordance with some embodiments.

FIG. 5A illustrates a delivery tube 502 of the apparatus 100 for fetal health monitoring, in accordance with some embodiments.

FIG. 5B is an enlarged view showing a portion “A” of FIG. 5A, in accordance with some embodiments.

FIG. 6 illustrates an extending of the portion of a skin 604 into a body cavity 110 of the apparatus 100 for fetal health monitoring, in accordance with some embodiments.

FIG. 7 illustrates a body portion 702 of the apparatus 100 for fetal health monitoring, in accordance with some embodiments.

FIG. 8 illustrates an attachment mechanism 808 of the apparatus 100 for fetal health monitoring, in accordance with some embodiments.

FIG. 9 illustrates a gap 902 results between the apparatus 100 and a skin 904, in accordance with some embodiments.

FIG. 10 illustrates an optical shield 1002 of the apparatus 100 for fetal health monitoring, in accordance with some embodiments

FIG. 11 illustrates a temperature sensor 1102 of the apparatus 100 for fetal health monitoring, in accordance with some embodiments.

FIG. 12 illustrates a tether 1202 of the apparatus 100 for fetal health monitoring, in accordance with some embodiments.

FIG. 13 illustrates a cross-sectional view of the apparatus 100 for fetal health monitoring including the delivery tube 502, in accordance with some embodiments.

FIG. 14 illustrates a clinical application of the apparatus 100, in accordance with some embodiments.

FIG. 15 illustrates a right side view of the apparatus 100 for fetal health monitoring including the delivery tube 502, in accordance with some embodiments.

FIG. 16 illustrates a perspective view of the apparatus 100 for fetal health monitoring including the delivery tube 502, in accordance with some embodiments.

FIG. 17 illustrates a left side view of the apparatus 100 for fetal health monitoring including the delivery tube 502, in accordance with some embodiments.

FIG. 18 illustrates a front view of the apparatus 100 for fetal health monitoring, in accordance with some embodiments.

FIG. 19 illustrates an exploded view of the apparatus 100 for fetal health monitoring, in accordance with some embodiments.

FIG. 20 illustrates a cross-sectional view of the apparatus 100 for fetal health monitoring including the delivery tube 502, in accordance with some embodiments.

FIG. 21 illustrates a rear view of the apparatus 100 for fetal health monitoring including the delivery tube 502, in accordance with some embodiments.

FIG. 22 illustrates a top view of the apparatus 100 for fetal health monitoring including the delivery tube 502, in accordance with some embodiments.

FIG. 23 illustrates an isometric view of the apparatus 100 for fetal health monitoring including the delivery tube 302, in accordance with some embodiments.

FIG. 24 is an illustration of an online platform 2400 consistent with various embodiments of the present disclosure.

FIG. 25 is a block diagram of a computing device 2500 for implementing the methods disclosed herein, in accordance with some embodiments.

FIG. 26 illustrates an apparatus 2600 for fetal health monitoring, in accordance with some embodiments.

FIG. 27 illustrates an apparatus 2700 for fetal health monitoring, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data there between corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

Pulse oximetry technology has been available since the 1940s. The physics of opto-electricity used in pulse oximetry has changed very little from inception to the present day. The pulse oximeter is a ubiquitous device used extensively in both outpatient and inpatient clinical settings as well as adult and pediatric medicine. The pulse oximetry technology is painless, noninvasive, and an effective method of determining the functional status of the respiratory and circulatory systems. The pulse oximeter's use is well established as a “standard of care” for establishing physiologic homeostasis.

At present, the best available method of monitoring fetal well-being (homeostasis) during labor and delivery relies on the rate and rhythm of the fetal heart. The tracing reveals patterns that have been interpreted as positive or negative, from which labor is managed. The above management scheme has not changed in theory since the early 1800s, when the difference in the maternal and fetal heart rate was first discovered (E. Kennedy, Rotunda Hospital, Dublin, Ireland). Measurement devices have been modernized and pattern interpretation has advanced, but the theory of predicting fetal distress has not. Many studies have confirmed the unreliability of fetal heart rate patterns to determine fetal distress. A false positive rate of 99% for fetal hypoxic ischemic encephalopathy, including Cerebral Palsy, has been determined by the American College of Obstetrics and Gynecology.

### Background & History of Electronic Fetal Heart and Oxygen Saturation Monitoring

Introduction of fetal pulse oximetry has prompted many interested observers to recall the introduction of electronic fetal heart rate monitoring that occurred approximately 40 years ago. There were great expectations that:
Electronic fetal heart rate monitoring would provide accurate information.
The information was of value in diagnosing fetal distress.
Possible to intervene to prevent fetal death or morbidity.
Continuous fetal heart rate monitoring was superior to intermittent monitoring.

When first introduced, the electronic fetal heart rate monitor consisted of an external ultrasound transducer on the mother's abdomen that transmitted the heart rate to a computer to produce a pattern for interpretation. Further advancements produced an internal monitoring system using a scalp screw type electrode, which was used primarily in complications of pregnancy. Gradually, continuous external monitoring came to be used during most labor. In 1978, estimated that nearly ⅔ of American women were being monitored electronically during labor, and in 1998, nearly 3.3 million American women, comprising 84% of all live births, underwent electronic fetal heart rate monitoring.

With the continuous use of the fetal monitor, there were questions about the efficacy, safety, and cost being voiced by the Office of Technology Assessment, the United States Congress, and the Centers for Disease Control and Prevention. Researchers have analyzed 158 reports and concluded that “the technical advances required in the demonstration that reliable recording could be done seem to have blinded most observers to the fact that the additional information will not necessarily produce better outcomes”. Researchers attributed the apparent lack of benefit to the imprecision of electronic monitoring to identify fetal distress. Moreover, increased usage was linked to more frequent cesarean deliveries.

### Fetal Heart Trace and Cardiotocography (CTG)

Cardiotocography has undergone little technological advancement over several decades. The fetal heart rate and the activity of the uterine muscle are detected by two transducers placed on the mother's abdomen, with one above the fetal heart to monitor heart rate, and the other at the fundus of the uterus to measure the frequency of contractions. Doppler ultrasound provides the information, which is recorded on a paper strip known as a cardiotocography (CTG). External fetal heart rate monitoring may be either continuous or intermittent. External tocography is useful for showing the beginning and end of contractions, as well as the frequency of the contractions, but not the strength of the contractions. The absolute values of pressure readings on an external tocometer are dependent on position and may lack sensitivity due to patient body habitus, a problem with fetal heart rate monitoring, as well. In cases where information on the strength or precise timing of contractions is needed, an internal tocodynamometer is more appropriate.

Internal cardiotocography uses an electronic transducer inserted into the uterus in proximity to the fetus and a spiral scalp electrode connected directly to the fetus. A wire electrode, sometimes called a spiral or scalp electrode, is attached to the fetal scalp through the cervical opening and is connected to the monitor. Internal monitoring provides a more accurate and consistent transmission of the fetal heart rate, and unlike external monitoring, the internal monitoring is not affected by factors such as movement or body habitus.

Internal monitoring may be used when external monitoring is inadequate or if closer surveillance is needed. Internal cardiotocography may only be used if the amniotic sac is ruptured (either spontaneously or artificially) and the cervix is open. To gauge the strength of contractions, a small catheter (called an intrauterine pressure catheter or IUPC) is passed into the uterus past the fetus. Combined with an internal fetal scalp electrode and an IUPC, the monitor may give a more precise reading of the baby's heart rate and the strength of contractions.

A typical CTG reading is printed on paper and may be stored on a computer for later reference. A variety of systems for centralized viewing of CTG have been installed in maternity hospitals in industrialized countries, allowing simultaneous monitoring of multiple tracings in one or more locations. Display of maternal vital signs, ST signals, and an electronic cardiotocogram is available in the majority of the systems. A few of the systems have incorporated computer analysis of cardiotocographic signals or combined cardiotocographic and ST data analysis.

### Fetal Scalp Stimulation

To supplement the information obtained using the fetal heart trace data by cardiotocography, which does not directly monitor the blood oxygen saturation level, the fetal scalp stimulation procedure is utilized. The fetal heart tracing and use of cardiotocography (CTG) were introduced in the late 1960s to recognize and respond to signs of intrapartum fetal distress. Fetal distress as indicated by the CTG was thought to be a direct indicator of intrauterine fetal hypoxia that might result in severe perinatal morbidity or even death. CTG is characterized by a high sensitivity, a low specificity, and a low positive predictive value for adverse outcomes. Thus, a normal, reassuring fetal heart tracing, a negative tracing is the only pattern with which the interpreter may reliably predict the outcome. Furthermore, the fetal heart tracing may also be associated with a high intra-and inter-observer variation, potentially leading to an unnecessary and inappropriately high operative delivery rate with risks for both the fetus and the mother. To improve the outcome and reduce interventions, various second-line tools have been suggested.

A fetal scalp stimulation (FSS) test was first described in 1936 by Sonntag and rests on the assumption that a reassuring fetus or a fetus with mild acidemia will respond to a certain stimulus by an increase in the heart rate.

Four methods for fetal stimulation are described in the literature: vibroacoustic, Allis clamp application, digital, and puncture of the scalp for fetal scalp blood sampling (FBS). The only published meta-analysis is based on 11 articles. All 11 studies had a small number of participants for each of the four tests. Only one article describes the stage of labor when the FSS test was conducted. Despite that, the authors of the meta-analysis argue for the use of FSS given the low likelihood of fetal acidemia when a negative test result is present, i.e., fetal response at stimulation.

Intrapartum fetal scalp blood sampling with measurement of scalp blood pH or lactate provides direct information about the acid-base status of the fetus. The internationally accepted cutoffs for normality are pH≥7.25 and lactate<4.2 mmol/L. From observational studies, there is evidence that fetal scalp blood sampling is associated with decreased operative deliveries (cesarean section) and perhaps also a reduction in severe neonatal acidosis. Review of clinical data indicates there is an association between the fetal ability to react to a scalp stimulus and fetal metabolism. However, the efficiency of an FSS test was too poor to rule in or rule out fetal hypoxia. Therefore, the use of the FSS test with caution is recommended, especially during the second stage, where the absence of accelerations also after provocation seems to be a normal phenomenon.

### Recommendations on Fetal Scalp Stimulation

In the presence of a fetal tracing that is interpreted as pathologic, fetal scalp stimulation during vaginal examination may be used to assess fetal health. The goal of the test is to induce an acceleration, or an increase in the fetal heart rate. A positive result would indicate a healthy fetus. If the test is negative, then proceed to the fetal blood sampling if the CTG remains pathologic.

Recommendations on Fetal Scalp Blood Sampling

Fetal scalp blood sampling is no longer conducted in most parts of the world. However, some information on how to conduct a sampling is provided below.

Do not carry out fetal blood sampling if there is an acute event (for example, cord prolapse, suspected placental abruption, or suspected uterine rupture) or the whole clinical picture indicates that the birth should be expedited or contraindications are present, including risk of maternal-to-fetal transmission of infection or risk of fetal bleeding disorders.

Be aware that for women with sepsis or significant meconium, fetal blood sample results may be falsely reassuring, and always discuss with a consultant obstetrician whether fetal blood sampling is appropriate and any results from the procedure if carried out.

Before carrying out or repeating fetal blood sampling, start conservative measures and offer digital fetal scalp stimulation. Only continue with fetal blood sampling if the cardiotocograph trace remains pathological. When considering fetal blood sampling, consider the woman's preferences and the whole clinical picture. When considering fetal blood sampling, explain the following to the woman and her birth companion(s):
Why the test is being considered and other options available, including the risks, benefits, and limitations of each.
The blood sample will be used to measure the level of acid in the baby's blood, which may help to show how well the baby is coping with labor.
The procedure will require her to have a vaginal examination using a device similar to a speculum.
A sample of blood will be taken from the baby's head by making a small scratch on the baby's scalp. The small scratch will heal quickly after birth, but there is a small risk of infection.
What the different outcomes of the test may be (normal, borderline, and abnormal) and the actions that will follow each result.
If a fetal blood sample may not be obtained but there are fetal heart rate accelerations in response to the procedure, the accelerations are encouraging and, in the above circumstances, expediting the birth may not be necessary.
If a fetal blood sample may not be obtained and the cardiotocograph trace has not improved, expediting the birth will be advised.
A caesarean section or instrumental birth (forceps or ventouse) may be advised, depending on the results of the procedure.

Do not take a fetal blood sample during or immediately after a prolonged deceleration. Take fetal blood samples with the woman in the left-lateral position. Use either pH or lactate when interpreting fetal blood sample results per the guideline.

If fetal blood sampling is attempted and a sample may not be obtained, but the associated fetal scalp stimulation results in a fetal heart rate acceleration, decide whether to continue the labor or expedite the birth in light of the clinical circumstances and in discussion with the woman and a senior obstetrician. If fetal blood sampling is attempted but a sample may not be obtained, and there has been no improvement in the cardiotocograph trace, expedite the birth.

### Background on Fetal Acidosis

Acidosis means a high hydrogen ion concentration in the tissues. Acidemia refers to a high hydrogen ion concentration in the blood and is the best proxy for tissue acidosis due to ease of measurement. The unit most used for acid-base status is pH, which is a log to base 10 of the reciprocals of the hydrogen ion concentration. Whereas blood pH may change quickly, tissue pH is more stable. The cut-off taken to define acidemia in adults is a pH of less than 7.36, but after labor and normal delivery, much lower values commonly occur in the fetus (pH 7.00), often with no subsequent deleterious effects. Studies looking at the pH of fetuses from cord blood samples taken antenatally and at delivery have established reference ranges.

Acidosis occurs when the tissue becomes hypoxic; unclear whether the consequences of the process are due primarily to the acidosis or the hypoxia. What has become clear over the past decade is that the outcomes resulting from hypoxia/acidosis are very different, depending on whether the process is acute or chronic. The normal human fetus is adapted to survive labor and has compensatory mechanisms that allow the fetus to withstand even severe hypoxia and acidosis for short periods of time.

In clinical practice, the level of arterial oxygenation may be measured either directly by an arterial blood gas sampling to measure both the partial pressure (PaO2) and percentage saturation (SaO2) of oxygen or indirectly by pulse oximetry (SpO2), meaning arterial oxygen saturation as noted SaO2. Provided that hemoglobin and circulatory function are normal, a patient's arterial oxygen saturation (measured either directly on arterial blood, (SaO2), or estimated by pulse oximetry, (SaO2)) gives information about the amount of oxygen that is available to the metabolizing tissues. The blood gas sampling measurements are directly related to pulse oximetry measurements (solid line normal patient, dotted line anemic patient). The fetal blood sample results of normal, borderline, and abnormal conditions include a pH value of 7.25 or above, 7.21 to 7.24, and 7.20 or below, respectively. The fetal blood sample results of normal, borderline, and abnormal conditions include a lactate level of 4.1 mmol/ L or below, 4.2 to 4.8 mmol/L, and 4.9 mmol/L or above, respectively.

The present disclosure relates to a Fetal Scalp Sensor Array (FSSA). Further, the present disclosure describes an anchored sensor system that may use a physical temporary anchoring system to attach to the scalp of the fetus intra-partum. FSSA may facilitate continuous fetal health monitoring. The physical temporary anchoring system may be similar to the fetal scalp electrode, which uses a pig-tail needle to attach to the scalp. Unlike the fetal scalp electrode, which is used to pick up a single electrical signal, FSSA may incorporate multiple sensors in the body of the device as well as use the needle for both anchoring and as an electrode, and in some versions, thermal pick up and fluid pick up. In some versions, some or all of the sensors may be in direct contact with the skin. In some versions, the sensors, other than the pigtail needle, may be recessed inward from the surface of the outer diameter of the housing, allowing for a cavity or gap to be present between the surface of the skin and the sensors.

The system may be connected to external monitors using one or more wire cables or a combination of wire cable and wireless communication, just wireless communication, or to an attached/integrated monitor.

The electrical signal picked up by the pigtail needle may be cross-compared in a time delineated method to the SpO2 pulse signal using the software of the system (E.g., the SpO2 pulse needs x beats to determine pulse rate, the data may be overlaid/compared to a “frame” delayed real-time electrical pick up). The pigtail needle may act as a thermal pick-up. The pigtail attachment needle might be hollow to pick up fluids for analysis (acid-base (pH)/metabolites/CO2). The wireless version may connect to an application residing on a phone, tablet, or other type of computer.

The device may comprise the following stacked layout incorporates the SpO2 sensor, temperature sensor, Bluetooth module (nRF52805), battery, and tether attachment, ensuring optimal functionality and manufacturability for a single-use, sterilized medical device. The device may be approximately an 8.0 mm diameter device.

### Layer 1: Sensor Base Layer

### Components

1. SpO2 Sensor (MAX30102EFD+T):
Mounted on the base PCB to ensure direct contact with the fetal scalp.
Optical window (transparent medical-grade silicone or polycarbonate) to protect the sensor.
2. Electrode Connection:
Spiral electrode for fetal heart rate monitoring, integrated into the housing base.
Low-resistance conductive material for accurate signal capture.
The tip of the spiral electrode is placed one millimeter above the surface of the case housing.
3. Tether Attachment:
Securely anchored tether point at the edge of the housing.
The threaded suture-type tether extends through the delivery sheath.

### Dimensions

Diameter: 7.5 mm (matching the device housing).
Height: ˜1.6 mm (sensor and PCB thickness).

### Layer 2: Processing and BLE Layer Components

1. Bluetooth Module (nRF52805):
Central placement on a small PCB with minimal external components.
Printed PCB antenna or a compact ceramic chip antenna integrated into the layer.
2. Temperature Sensor:
Mounted on the same PCB, adjacent to the SpO2 sensor, for thermal monitoring.
3. Signal Processing:
ARM Cortex-M0 microcontroller (embedded in nRF52805) for signal acquisition and Bluetooth transmission.
4. Power Distribution:
Connects directly to the battery above via vertical or flex PCB.

### Dimensions

Diameter: 7.5 mm.
Height: ˜1.5 mm (Bluetooth module, antenna, and sensor integration).

### Layer 3: Power Source (Battery Layer) Components:

1. Battery:
Primary lithium coin cell (e.g., SR614SW).
Provides 1.55 V and ˜11 mAh capacity for 24+ hours of operation.
2. Battery Housing:
Insulated compartment to protect the battery from short circuits or environmental factors.
3. Connections:
Vertical traces or flex PCB linking battery output to the BLE and sensor PCBs.

### Dimensions

Diameter: 7.5 mm.
Height: ˜1.5 mm (battery thickness and insulation).

### Total Dimensions

Diameter: 7.5 mm (all layers).
Height: ˜4.8 mm (sensor+BLE+battery stack).

### Layer Stacking Order and Functionality

Base Layer (Layer 1): Function: Direct patient interface with SpO2 and heart rate sensing.
Intermediate Layer (Layer 2): Function: Signal processing, Bluetooth communication, and temperature sensing.
Top Layer (Layer 3): Function: Power supply and connection to the BLE and sensor layers.

Further, the SpO2 sensor might not be flush with the housing that the SpO2 sensor sits in, and the SpO2 sensor might be recessed. But the skin in that area might pucker up and touch the sensor ideally. If the skin doesn't pucker up, maybe the housing edge is flexible so that the housing seals in the area where the SpO2 sensor is, and may allow the sensor to touch the skin.

Further, the sensor might be spring-loaded so that the spring pushes the sensor against the skin.

Labeling: Use a QR code to pair the FSSA Bluetooth to Monitor devices (iPhone, Android) with the FSSA App. To connect, launch the app. The app will ask to view the existing connection or make a new connection. Choosing to make a new will open the camera in order to scan the QR.

The present disclosure describes an anchored fetal scalp sensor array (FSSA) that addresses long-standing limitations in intrapartum fetal monitoring. Further, the FSSA may include a temporary anchoring system similar to, but technologically more advanced than, conventional fetal scalp electrodes. Unlike traditional electrodes that capture only a single electrical signal, the FSSA may incorporate multiple types of sensors, thereby providing multi-parametric monitoring of fetal health. The technical problem addressed is the limited fidelity and scope of existing fetal heart rate and cardiotocographic systems, which may be plagued by false positives, poor specificity, and susceptibility to maternal body habitus.

Further, the spiral or pigtail needle used for anchoring may simultaneously act as an electrode, a thermal pickup, or a hollow conduit for sampling fetal fluids, solving the technical problem of requiring multiple invasive devices for discrete functions by converging multiple sensing modalities into a single device. For example, the hollow pigtail needle may enable real-time collection of fetal fluids for acid-base analysis or metabolite profiling, improving neonatal diagnostic technology.

Further, the FSSA may integrate a recessed or spring-loaded SpO₂ sensor, allowing consistent skin contact despite variations in fetal scalp topography or maternal uterine pressure. The technical problem of inconsistent optical coupling in pulse oximetry sensors is thus addressed. For instance, a flexible housing edge may deform to create a sealed pocket, ensuring that the recessed sensor remains in contact with the fetal scalp. Alternatively, spring loading may mechanically bias the sensor to maintain constant contact. The FSSA may improve optical biosensing technology by enhancing the robustness of real-time fetal oxygenation measurements.

Further, the device may provide cross-comparison of time-delineated electrical signals (ECG) with pulse-derived signals (SpO₂). Further, the device may address the technical problem of signal noise and false readings by enabling software-based synchronization across modalities. For instance, the ECG beat detected by the pigtail electrode may be overlaid against the delayed SpO₂ waveform to confirm temporal alignment. Further, the device may improve biomedical signal processing technology by reducing inter-sensor noise mismatches.

Further, the FSSA may be designed as a stacked three-layer system—sensor base, processing/Bluetooth layer, and power layer—within a 7.5 mm diameter, 4.8 mm high device. The compact stacking may solve the technical problem of incorporating multiple functionalities into a sterilizable, single-use device without increasing invasiveness. For example, the base may contain the SpO₂ sensor and electrode, the intermediate layer may host an ARM Cortex-M0-based Bluetooth module for wireless communication, and the top layer may house a miniaturized primary lithium coin cell battery. Further, the FSSA may improve implantable and wearable medical device miniaturization technology.

Further, wireless communication may be facilitated by BLE (Bluetooth Low Energy) via ceramic antennas or printed PCB antennas, enabling the device to stream data to smartphones, tablets, or clinical monitors. The technical problem addressed is the tethered, wired dependency of traditional fetal monitoring devices, which limit maternal mobility during labor. By using BLE, the device may improve wireless medical telemetry technology.

Further, the FSSA may integrate QR code-based pairing for streamlined setup with clinical monitors or consumer devices. The technical problem solved is the complex and error-prone device pairing process in high-stress clinical settings. For instance, the clinician may scan a QR code embedded on the device, triggering instant authentication and secure pairing. The FSSA may improve device usability and wireless security technology in medical monitoring systems.

Further, the FSSA may incorporate embedded or cloud-based AI algorithms to analyze fetal heart rate, SpO₂, and biochemical signals in real time. The technical problem addressed is the high inter-and intra-observer variability in CTG interpretation. By training models on large multi-center datasets, the system may automatically classify fetal states (normal, borderline, pathological) with higher accuracy. For example, the AI may learn to differentiate between benign decelerations and true hypoxic patterns, thereby improving decision support in obstetric monitoring technology.

Further, the FSSA may extend beyond traditional red/infrared SpO₂ sensing by including green or near-infrared wavelengths. The technical problem addressed is the difficulty of obtaining reliable oxygenation signals in low-perfusion conditions. For instance, multi-spectral data fusion may compensate for signal dropouts, improving optical biosensing technology in low-signal environments.

Further, the FSSA may wirelessly synchronize with maternal uterine contraction sensors, such as intrauterine pressure catheters or wearable abdominal bands. The technical problem solved is the lack of contextualization between fetal stress responses and maternal contractions. For example, the system may calculate contraction-stress indices by overlaying fetal oxygen drops with contraction peaks. Further, the FSSA may improve maternal-fetal monitoring system integration technology.

Further, the hollow pigtail needle may be coupled with a microfluidic lab-on-chip cartridge that may perform real-time biochemical assays, such as lactate, pH, or CO₂ quantification. The technical problem addressed is the delay and invasiveness of traditional fetal scalp blood sampling. For example, capillary action may draw fetal interstitial fluid into an on-chip electrochemical sensor array. The FSSA may improve point-of-care diagnostics technology.

Further, the FSSA may upload encrypted data to secure hospital or cloud servers for remote supervision by specialists. The technical problem addressed is the limited availability of skilled obstetricians in smaller clinical centers. For instance, a rural hospital may use the FSSA to transmit continuous fetal monitoring data to a tertiary care center, enabling remote intervention recommendations. Further, the FSSA may improve telemedicine and remote obstetric monitoring technology.

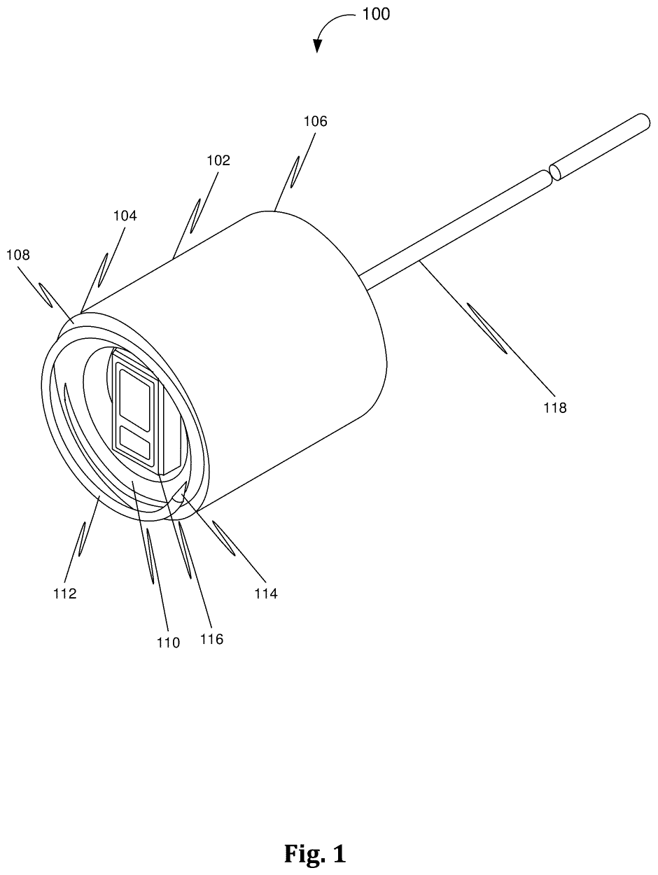

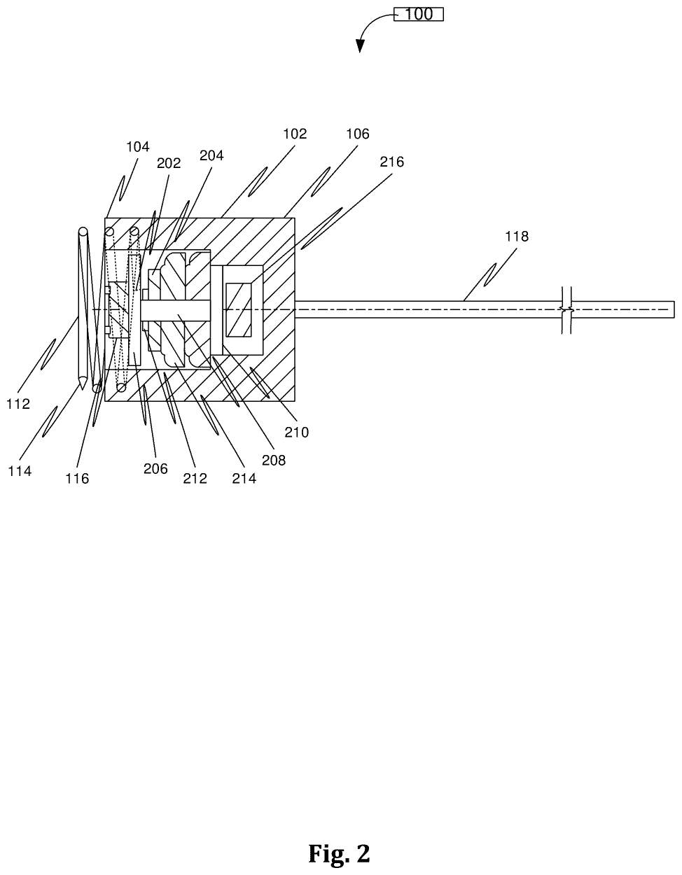

FIG. 1 illustrates an apparatus 100 for fetal health monitoring, in accordance with some embodiments. Further, the apparatus 100 may include a body 102 extending between a first body end 104 and a second body end 106. Further, the body 102 includes a body cavity 110. Further, the first body end 104 defines a body opening 108 leading into the body cavity 110. Further, the body 102 may be configured to be inserted into one or more user body cavities of one or more users. Further, the apparatus 100 may include an electrode 112 extending between a first electrode end 202 and a second electrode end 114. Further, the electrode 112 may be helically extending from the body 102. Further, the first electrode end 202 may be attached to the body 102, as shown in FIG. 2, and the second electrode end 114 may be free. Further, the second electrode end 114 may be configured to be inserted into one or more body parts of a fetus for securing the apparatus 100 to the one or more body parts based on one or more actions receivable on the body 102. Further, the electrode 112 may be configured for detecting one or more electrical signals associated with the one or more body parts of the fetus based on the inserting of the second electrode end 114 into the one or more body parts of the fetus. Further, the one or more electrical signals may be associated with one or more first physiological parameters of the fetus. Further, the apparatus 100 may include one or more sensors 116 disposed in the body cavity 110. Further, the one or more sensors 116 may be configured for detecting one or more second physiological parameters associated with the fetus. Further, the apparatus 100 may include a processing device 206 disposed within the body 102, as shown in FIG. 2. Further, the processing device 206 may be communicatively coupled with the electrode 112 and the one or more sensors 116. Further, the processing device 206 may be configured for generating one or more first physiological data based on the detecting of the one or more electrical signals. Further, the processing device 206 may be configured for generating one or more second physiological data based on the detecting of the one or more second physiological parameters. Further, the apparatus 100 may include a communication device 212 disposed within the body 102, as shown in FIG. 2. Further, the communication device 212 may be communicatively coupled with the processing device 206. Further, the communication device 212 may be configured for transmitting the one or more first physiological data and the one or more second physiological data to one or more external devices.

FIG. 2 illustrates a cross-sectional view of the apparatus 100 for fetal health monitoring, in accordance with some embodiments. Further, the cross-sectional view of the apparatus 100 shows the processing device 206, the first electrode end 202, and the communication device 212.

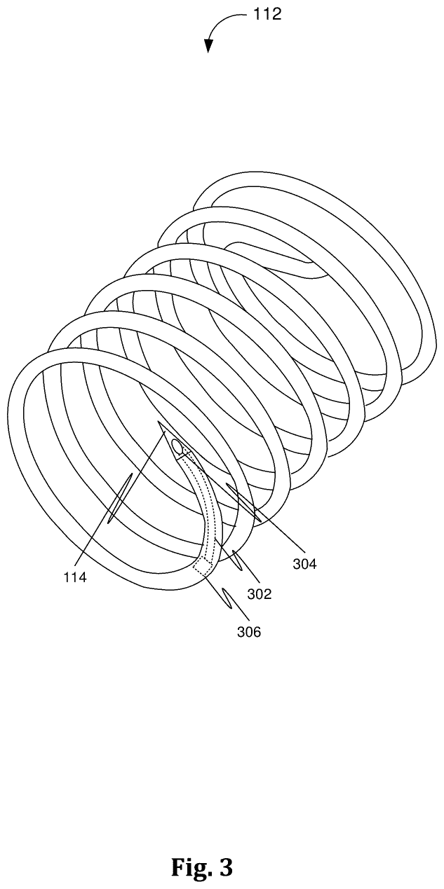

FIG. 3 illustrates the electrode 112 of the apparatus 100 for fetal health monitoring, in accordance with some embodiments. Further, the electrode 112 further includes an electrode cavity 302 extending from the second electrode end 114. Further, the second electrode end 114 defines an electrode opening 304 leading into the electrode cavity 302. Further, the electrode 112 further includes a sample collecting mechanism 306 coupled with the electrode cavity 302. Further, the sample collecting mechanism 306 may be configured to be activated. Further, the sample collecting mechanism 306 may be configured for collecting one or more samples associated with the fetus based on the inserting of the body 102 into the one or more user body cavities of the one or more users after the activating of the sample collecting mechanism 306. Further, the electrode 112 may be configured for storing the one or more samples in the electrode cavity 302 based on the collecting of the one or more samples.

In some embodiments, the sample collecting mechanism 306 may include a plunger.

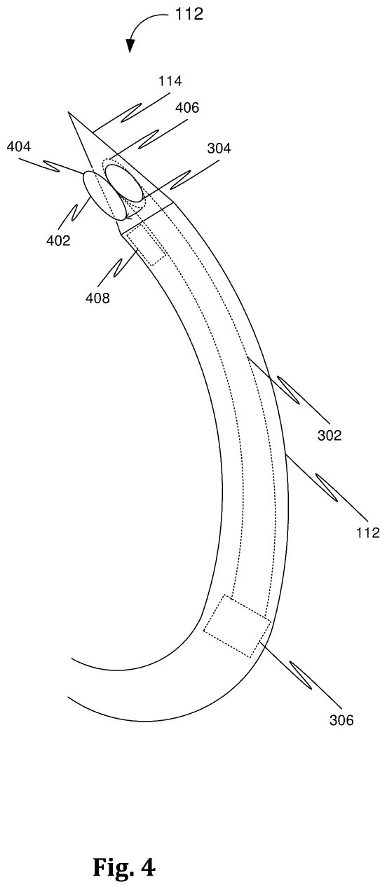

FIG. 4 illustrates the electrode 112 of the apparatus 100 for fetal health monitoring, in accordance with some embodiments. Further, the electrode 112 further includes a lid 402 coupled to a portion of the electrode 112. Further, the lid 402 may be configured to be transitioned between a closed position 406 and an open position 404. Further, the lid 402 covers the electrode opening 304 in the closed position 406, and the lid 402 uncovers the electrode opening 304 in the open position 404.

In some embodiments, the electrode 112 further includes a lid transitioning mechanism 408, as shown in FIG. 4. Further, the lid transitioning mechanism 408 may be configured for transitioning between a first state and a second state. Further, the lid 402 transitions to the closed position 406 may be based on the transitioning of the lid transitioning mechanism 408 from the first state to the second state. Further, the lid 402 transitions to the open position 404 may be based on the transitioning of the lid transitioning mechanism 408 from the second state to the first state. Further, the collecting of the one or more samples may be further based on the transitioning of the lid transitioning mechanism 408 from the second state to the first state.

In some embodiments, the lid transition mechanism 408 may include one or more of an actuator, a spring-loaded slider, a rotational hinge, and a twist lock system.

In some embodiments, the communication device 212 may be configured for receiving an activating command for the activating of the sample collecting mechanism 306. Further, the processing device 206 may be further configured for activating the sample collecting mechanism 306 based on the activating command.

In some embodiments, the sample collecting mechanism 306 may be configured to be deactivated. Further, the deactivating of the sample collecting mechanism 306 halts the collecting of the one or more samples.

In some embodiments, the communication device 212 may be configured for receiving a deactivation command for the deactivating of the sample collecting mechanism 306. Further, the processing device 206 may be further configured for deactivating the sample collecting mechanism 306 based on the deactivating command.

In some embodiments, the lid transitioning mechanism 408 may be operatively coupled with the sample collecting mechanism 306. Further, the transitioning of the lid transitioning mechanism 408 from the second state to the first state may be based on the activating of the sample collecting mechanism 306.

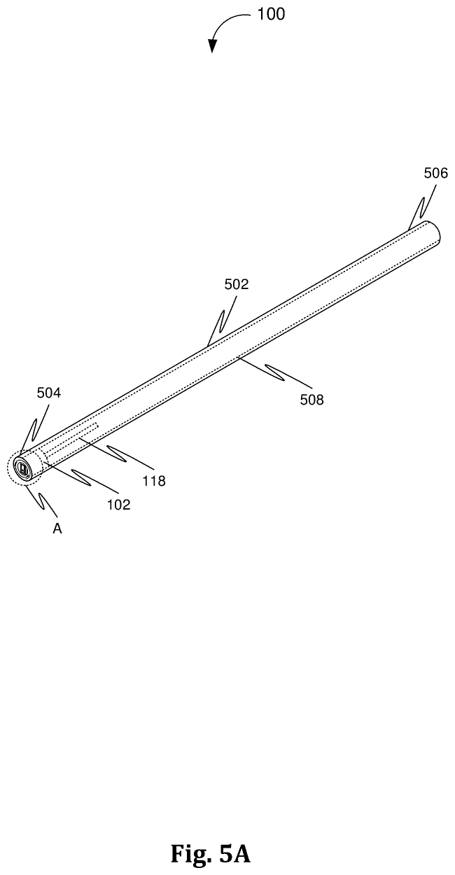

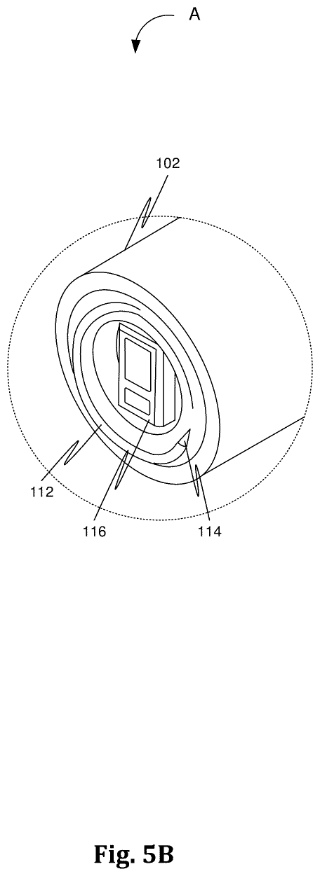

FIG. 5A illustrates a delivery tube 502 of the apparatus 100 for fetal health monitoring, in accordance with some embodiments. Further, the apparatus 100 may further include a delivery tube 502 extending between a first tube end 504 and a second tube end 506. Further, the delivery tube 502 includes a tube cavity 508 extending between the first tube end 504 and the second tube end 506. Further, the body 102 may be disposed in the tube cavity 508 at the first tube end 504. Further, the first tube end 504 may be inserted into the one or more user body cavities. Further, the first tube end 504 may be configured to be positioned in one or more positions in the one or more user body cavities in relation to the fetus after the inserting of the first tube end 504. Further, the inserting of the second electrode end 114 into the one or more body parts of the fetus may be based on the positioning of the first tube end 504 in the one or more positions. Further, the one or more action receivables on the body 102 may be applied using the delivery tube 502.

FIG. 5B is an enlarged view showing a portion “A” of FIG. 5A, in accordance with some embodiments. Further, the delivery tube 502 may be detached from the body 102 after the inserting of the second electrode end 114 into the one or more body parts of the fetus. Further, the detaching of the delivery tube 502 may be based on one or more second actions applied on the delivery tube 502.

In some embodiments, the apparatus 100 may further include a printed circuit board (PCB) 208 disposed in the body cavity, as shown in FIG. 2. Further, the PCB 208 includes each of the processing device 206 and the communication device 212. Further, each of the one or more sensors 116 and the first electrode end 202 of the electrode 112 may be mounted on the PCB 208.

In some embodiments, the PCB 208 may include a horizontal portion, a first vertical portion, and a second vertical portion 210, as shown in FIG. 2. Further, the processing device 206, the communication device 212, the one or more sensors 116, and the electrode 112 may be mounted on the first vertical portion. Further, a vertical portion of PCB 208 extends into the body cavity 110.

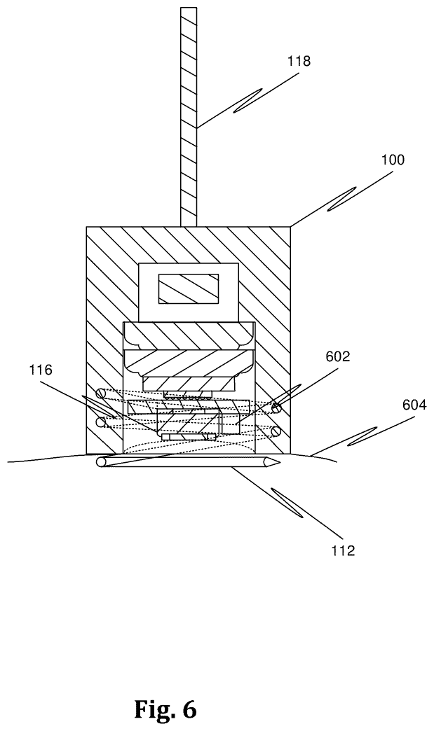

FIG. 6 illustrates an extending of a portion of the skin 604 into the body cavity 110 of the apparatus 100 for fetal health monitoring, in accordance with some embodiments. Further, the one or more sensors 116 disposed in the body cavity 110 may be recessed inward in relation to the body opening 108. Further, the apparatus 100 further includes a suction mechanism 602 disposed in the body cavity 110. Further, the suction mechanism 602 may be configured for extending a portion of a skin 604 of the one or more body parts into the body cavity 110 based on the securing of the apparatus 100 to the one or more body parts. Further, the extending of the portion of the skin 604 into the body cavity 110 for establishing a direct contact of the portion of the skin 604 with one or more sensing elements of the one or more sensors 116. Further, the detecting of the one or more second physiological parameters may be further based on the establishing of the direct contact of the portion of the skin 604 with the one or more sensing elements.

In some embodiments, the suction mechanism 602 may include a micro suction nozzle.

In some embodiments, the suction mechanism 602 may include a negative pressure channel.

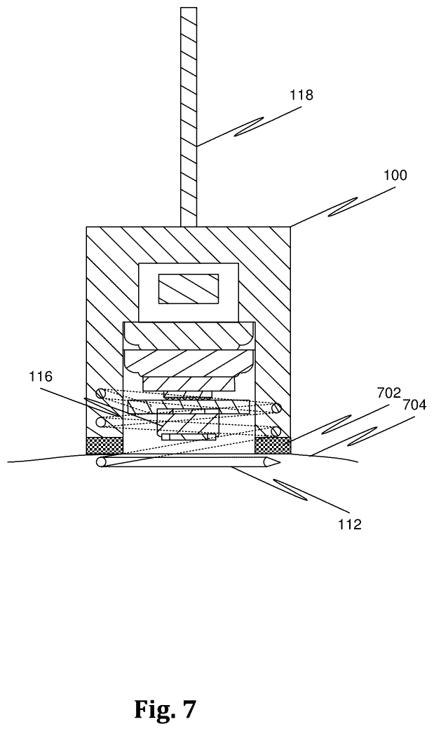

FIG. 7 illustrates a body portion 702 of the apparatus 100 for fetal health monitoring, in accordance with some embodiments. Further, the body 102 includes a body portion 702 defining the body cavity 110. Further, the body portion 702 may be configured to be deformed. Further, the one or more sensors 116 disposed in the body cavity 110 may be recessed inward in relation to the body opening 108. Further, the deforming of the body portion 702 after the securing of the apparatus 100 to the one or more body parts makes one or more sensing elements of the one or more sensors 116 to establish a direct contact with a portion of a skin 704 of the one or more body parts. Further, the detecting of the one or more second physiological parameters may be further based on the establishing of the direct contact of the portion of the skin 704 with the one or more sensing elements.

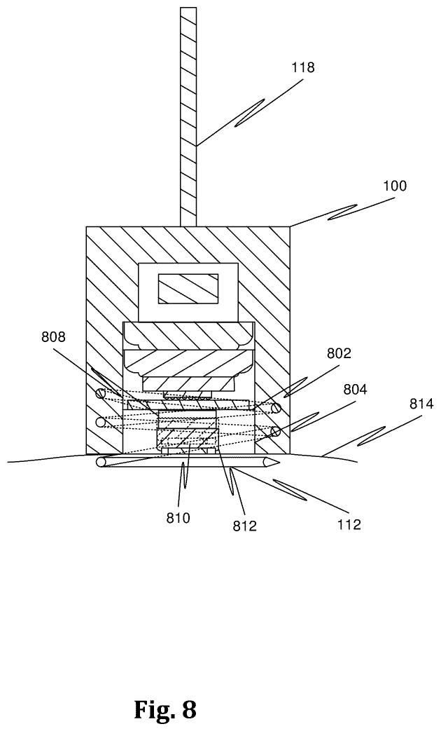

FIG. 8 illustrates an attachment mechanism of the apparatus 100 for fetal health monitoring, in accordance with some embodiments. Further, the body 102 includes an inner base surface 802 and an inner side surface 804 defining the body cavity 110. Further, the inner side surface 804 extends from the inner base surface 802 towards the body opening 108. Further, the one or more sensors 116 may be attached to the inner base surface 802 defining the body cavity 110 using an attachment mechanism 808. Further, the securing of the apparatus 100 to the one or more body parts results in a gap between a portion of a skin 814 of the one or more body parts and the inner base surface 802. Further, the attachment mechanism 808 may be configured for retractably extending the one or more sensors 116 in the gap for transitioning the one or more sensors 116 between an extended position 812 and a retracted position 810. Further, the one or more sensors 116 may be recessed inward in relation to the body opening 108 in the retracted position 810. Further, one or more sensing elements of the one or more sensors 116 may be in a direct contact with the portion of the skin 814 in the extended position 812. Further, the detecting of the one or more second physiological parameters may be further based on establishing of the direct contact of the portion of the skin 814 with the one or more sensing elements.

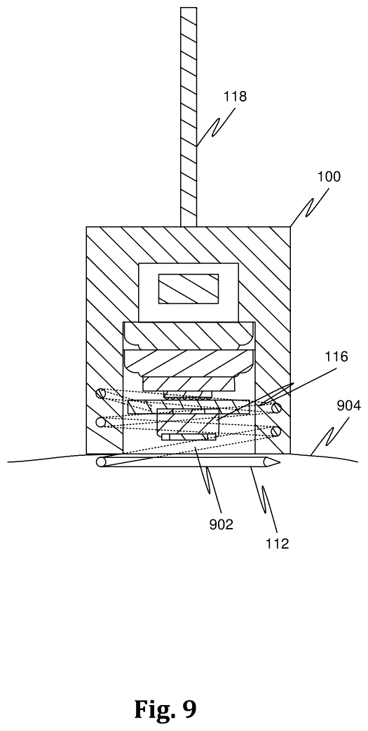

In some embodiments, the one or more sensors 116 disposed in the body cavity 110 may be recessed inward in relation to the body opening 108. Further, the securing of the apparatus 100 to the one or more body parts results in a gap 902, as shown in FIG. 9, between a portion of a skin 904 of the one or more body parts and one or more sensing elements of the one or more sensors 116. Further, the detecting of the one or more second physiological parameters includes performing one or more sensing operations by the one or more sensors 116 using the one or more sensing elements without a direct contact between the one or more sensing elements and the portion of the skin 904 of the one or more body parts.

FIG. 9 illustrates the gap 902 results between the apparatus 100 and the skin 904, in accordance with some embodiments. Further, the apparatus 100 may further include one or more batteries 214 disposed within the body 102, as shown in FIG. 2. Further, the one or more batteries 214 may be electrically coupled with the PCB 208. Further, each of the processing device 206, the communication device 212, and the one or more sensors 116 may be electrically powered.

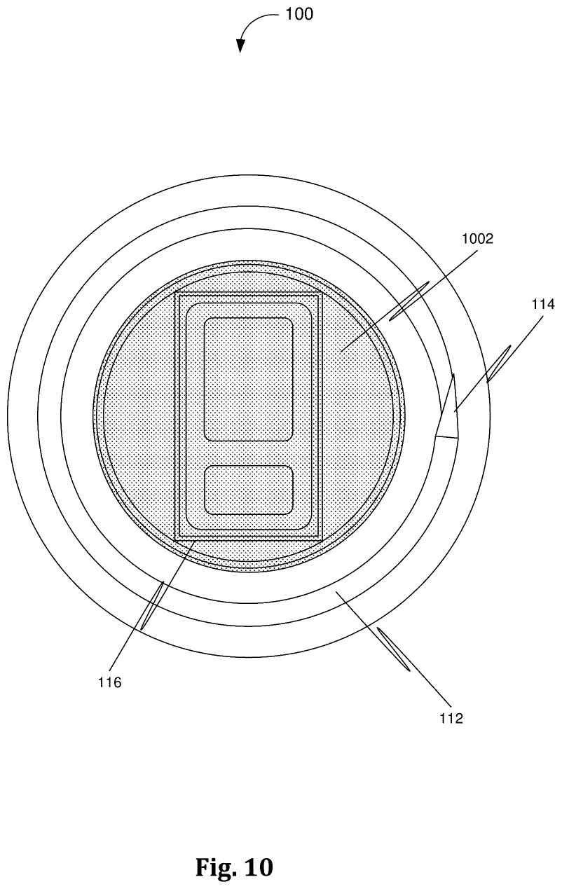

FIG. 10 illustrates an optical shield 1002 of the apparatus 100 and the skin 904, in accordance with some embodiments. Further, the body 102 includes an optical shield 1002 disposed on the body 102. Further, the optical shield 1002 covers the body opening 108 of the body 102. Further, the one or more sensors 116 may be configured for performing one or more sensing operations through the optical shield 1002. Further, the detecting of the one or more second physiological parameters associated with the fetus may be further based on the performing of the one or more sensing operations.

In some embodiments, the optical shield 1002 may be configured to minimize ambient light interference from a maternal environment.

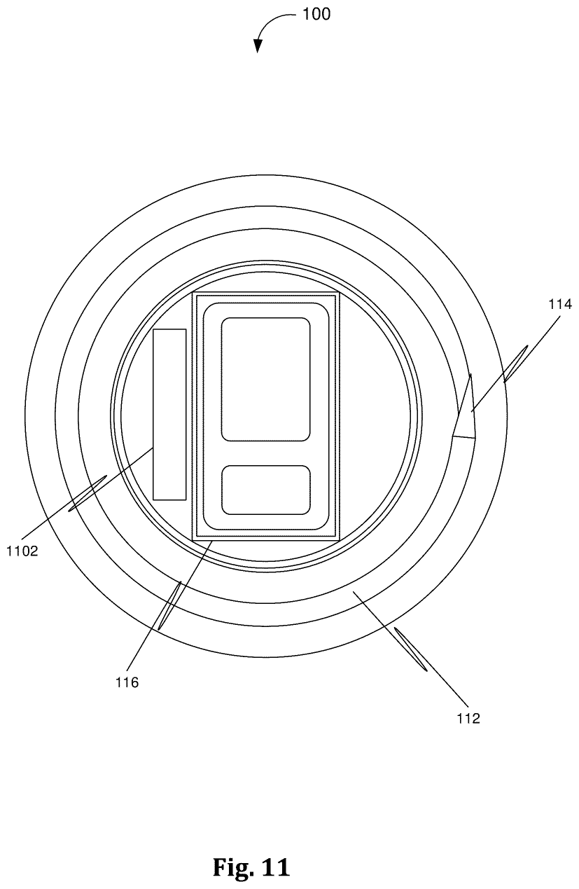

FIG. 11 illustrates a temperature sensor 1102 of the apparatus 100 for fetal health monitoring, in accordance with some embodiments. Further, the apparatus 100 may further include a temperature sensor 1102 disposed in the body cavity 110. Further, the temperature sensor 1102 may be placed adjacent to the one or more sensors 116 in the body cavity 110. Further, the temperature sensor 1102 may be configured for detecting a temperature of the fetus. Further, the temperature sensor 1102 may be mounted on the PCB 208. Further, the processing device 206 may be communicatively coupled with the temperature sensor 1102. Further, the processing device 206 may be further configured for generating a temperature data based on the detecting of the temperature of the fetus. Further, the communication device 212 may be further configured for transmitting the temperature data to the one or more external devices.

In some embodiments, the apparatus 100 may further include an insulation disk 204 disposed within the body 102, as shown in FIG. 2. Further, the insulation disk 204 may be disposed between the one or more batteries 214 and two or more components. Further, the insulation disk 204 may be configured for providing an insulation between the one or more batteries 214 and the two or more components. Further, the two or more components include one or more of the one or more sensors 116, the electrode 112, the processing device 206, and the communication device 212.

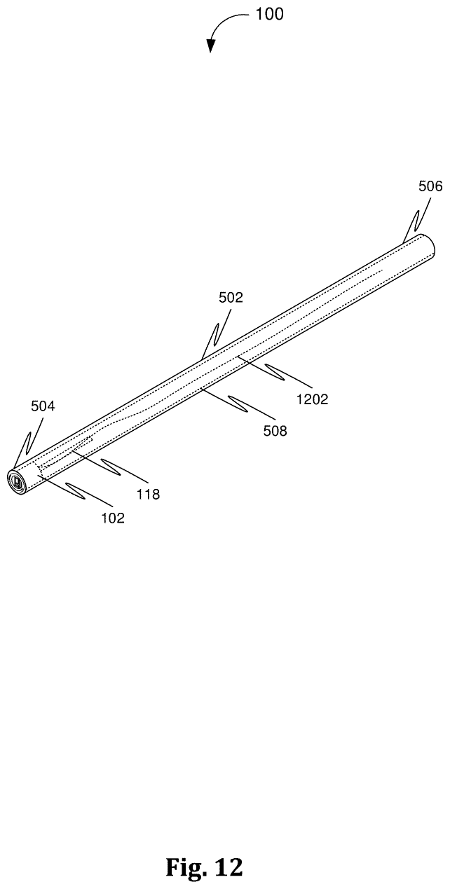

FIG. 12 illustrates a tether 1202 of the apparatus 100 for fetal health monitoring, in accordance with some embodiments. Further, the apparatus 100 may further include a tether 1202 extending between a first tether end and a second tether end. Further, the first tether end of the tether 1202 may be attached to the body 102 at the second body end 106. Further, the tether 1202 extends through the delivery tube 502. Further, the tether 1202 may be configured to facilitate controlling a position of the body 102 on the one or more body parts.

Further, in some embodiments, the processing device 206 may be further configured for comparing the one or more first physiological data with the one or more second physiological data using a time-delineated method. Further, the processing device 206 may be further configured for obtaining a comparison data based on the comparing of the one or more first physiological data with the one or more second physiological data. Further, the communication device 212 may be further configured for transmitting the comparison data to the one or more external devices.

Further, in some embodiments, the processing device 206 may be further configured for comparing the one or more first physiological data, the one or more second physiological data, and an external CTG data to obtain an additional comparison data. Further, the communication device 212 may be further configured for transmitting the additional comparison data to the one or more external devices.

Further, in some embodiments, the communication device 212 may be further configured for receiving the external CTG data from the one or more external devices.

In some embodiments, the deforming of the body portion 702 after the securing of the apparatus 100 to the one or more body parts includes deforming the body portion 702 after the securing of the apparatus 100 to the one or more body parts for creating a seal between the portion of the skin 704 of the one or more body parts and the one or more sensing elements of the one or more sensors 116. Further, the establishing of the direct contact may be based on the creating of the seal.

In some embodiments, the attachment mechanism 808 may be based on one or more springs. Further, the one or more sensors 116 may be attached to the inner base surface 802 using the one or more springs. Further, the one or more sensors 116 may be retractably extended in the gap using the one or more springs.

In some embodiments, the apparatus 100 may be secured to the one or more body parts of the fetus during an intrapartum period of the one or more users. Further, the one or more users include the fetus.

In some embodiments, the one or more sensors 116 include a peripheral oxygen saturation (SpO₂) sensor. Further, the one or more second physiological parameters include an oxygen saturation level of the fetus.

In some embodiments, the one more second physiological data include a fetal heat rata and an arterial oxygen saturation (SpO₂).

In some embodiments, the one or more sensors 116 may include one or more optical emitters and one or more optical detectors. Further, the detecting of the one or more second physiological parameters includes emitting a light towards the one or more body parts using the one or more optical emitters. Further, the detecting of the one or more second physiological parameters further includes receiving a reflected light from the one or more body parts using the one or more optical detectors.

In some embodiments, the light may be characterized by a plurality of wavelengths including approximately 660 nanometers and approximately 940 nanometers. Further, the one or more second physiological parameters correspond to photoplethysmographic signals.

In some embodiments, the processing device 206 may be configured for filtering the one or more second physiological data to obtain a processed second physiological data. Further, the processed second physiological data lacks a noise data corresponding to maternal pulse interference. Further, the transmitting of the one or more first physiological data and the one or more second physiological data comprises transmitting the one or more first physiological data and the processed second physiological data to the one or more external devices.

In some embodiments, the processing device 206 may be configured to use an artifact rejection algorithm for the filtering of the one or more second physiological data.

In some embodiments, the one or more first physiological parameters include a heart rate of the fetus.

In some embodiments, the electrode 112 includes a conductive material possessing a low electrical resistance property.

In some embodiments, the electrode 112 includes a spiral needle. Further, the second electrode end 114 includes a needle tip. Further, the needle tip may be positioned away from the body opening 108 at a distance of one millimeter.

In some embodiments, the tether 1202 includes a threaded suture tether.

In some embodiments, the communication device 212 includes a Bluetooth module comprising one of a printed circuit board (PCB) antenna and a ceramic chip antenna.

In some embodiments, the processing device 206 includes a microcontroller. Further, the microcontroller may be embedded in the Bluetooth module.

In some embodiments, the optical shield 1002 includes one or more of a medical-grade silicone material and a medical-grade polycarbonate material.

In some embodiments, the optical shield 1002 may be characterized by one or more of an optical transparency and a medical-grade property.

In some embodiments, the one or more batteries 214 include a primary lithium coil cell.

In some embodiments, the providing of the insulation between the one or more batteries 214 and the two or more components may be for protecting the one or more batteries 214 from a short circuit.

In some embodiments, the one or more batteries 214 may be characterized by one or more of twenty-four hours of operation time, eleven milliampere-hour capacity, and one and fifty-five hundred voltages.

In some embodiments, the one or more external devices may be further configured for scanning a quick response (QR) code. Further, the QR code corresponds to a unique identifier of the apparatus 100. Further, the one or more external devices may be further configured for establishing a connection between the apparatus 100 and the one or more external devices based on the scanning of the QR code. Further, the transmitting of the one or more first physiological data and the one or more second physiological data to the one or more external devices may be further based on the establishing of the connection between the apparatus 100 and the one or more external devices.

In some embodiments, the one or more external devices may be further configured for executing one or more applications. Further, the scanning of the QR code may be further based on the executing of the one or more applications.

In some embodiments, the one or more external devices may be further configured for comparing at least one of the one or more first physiological data and the one or more second physiological data with one or more predetermined thresholds. Further, the one or more external devices may be further configured for generating an alert based on the comparing of at least one of the one or more first physiological data and the one or more second physiological data with one or more predetermined thresholds.

In some embodiments, the apparatus 100 may be communicatively coupled with the one or more external devices using one or more wire cables.

In some embodiments, the apparatus 100 may be communicatively coupled with the one or more external devices through a wireless communication technology.

In some embodiments, the one or more first physiological data indicate a heart rate of the fetus. Further, the one or more second physiological data indicate an oxygen saturation level of the fetus. Further, the comparison data includes an overlay of the one or more second physiological data on the one or more first physiological data for indicating a pulse rate of the fetus.

In some embodiments, the collecting of the one or more samples may be for one or more analysis. Further, the one or more analyses include one or more of an acid-base analysis, a pH analysis, a metabolites analysis, and a carbon dioxide analysis.

In some embodiments, the one or more samples include blood.

In some embodiments, the one or more samples include an amniotic fluid associated with the fetus.

In some embodiments, the body portion 702 defining the body cavity 110 includes a flexible material.

In some embodiments, the one or more body parts include a scalp of the fetus.

In some embodiments, the one or more body parts include a skin of the scalp.

In some embodiments, the electrode 112 includes a pigtail needle.

In some embodiments, the apparatus 100 may further include a magnetic reed switch 216 disposed within the body 102, as shown in FIG. 2. Further, the magnetic reed switch 216 may be configured to be transitioned between an open state and a closed state based on a presence of a magnetic field. Further, the magnetic reed switch 216 may be configured for allowing an electrical energy to flow from the one or more batteries 214 to the two or more components in the closed state. Further, the magnetic reed switch 216 may be associated with the closed state during a presence of the magnetic field. Further, the magnetic reed switch 216 may be configured for restricting the electrical energy to flow from the one or more batteries 214 to the two or more components in the open state. Further, the magnetic reed switch 216 may be associated with the open state during an absence of the magnetic field.

In some embodiments, the PCB 208 includes one of a vertical printed circuit board (PCB) and a flexible printed circuit board (PCB).

In some embodiments, the one or more actions include a first rotation of the delivery tube 502 in a first direction. Further, the one or more second actions include a second rotation of the delivery tube 502 in a second direction. Further, the first direction may be different from the second direction.

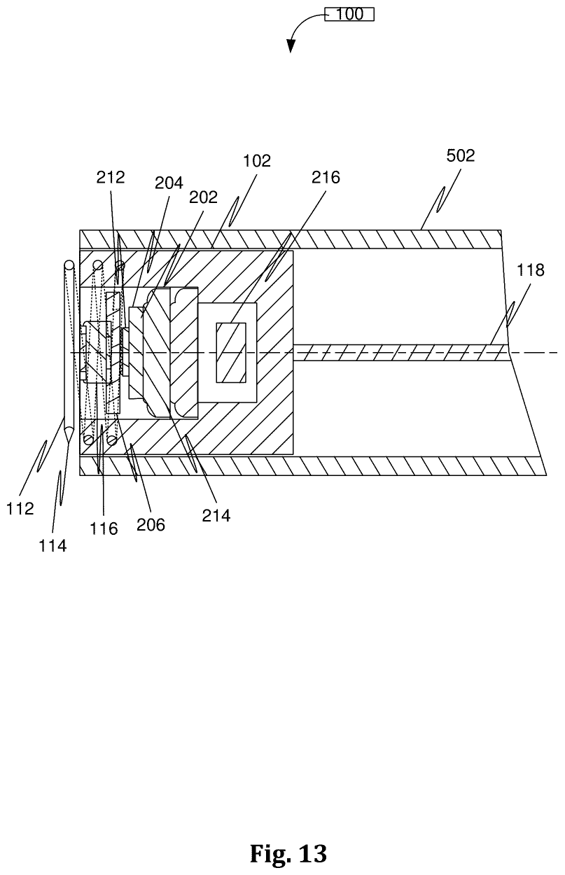

FIG. 13 illustrates a cross-sectional view of the apparatus 100 for fetal health monitoring including the delivery tube 502, in accordance with some embodiments. Further, the cross-sectional view of the apparatus 100 including the delivery tube 502 shows the processing device 206, the communication device 212, the first electrode end 202, and the insulation disk 204.

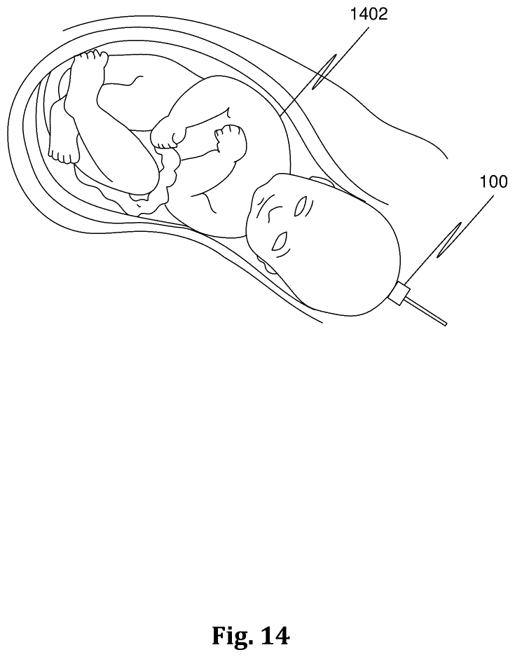

FIG. 14 illustrates a clinical application of the apparatus 100, in accordance with some embodiments. Further, the apparatus 100 may be inserted into a scalp of a fetus 1402.

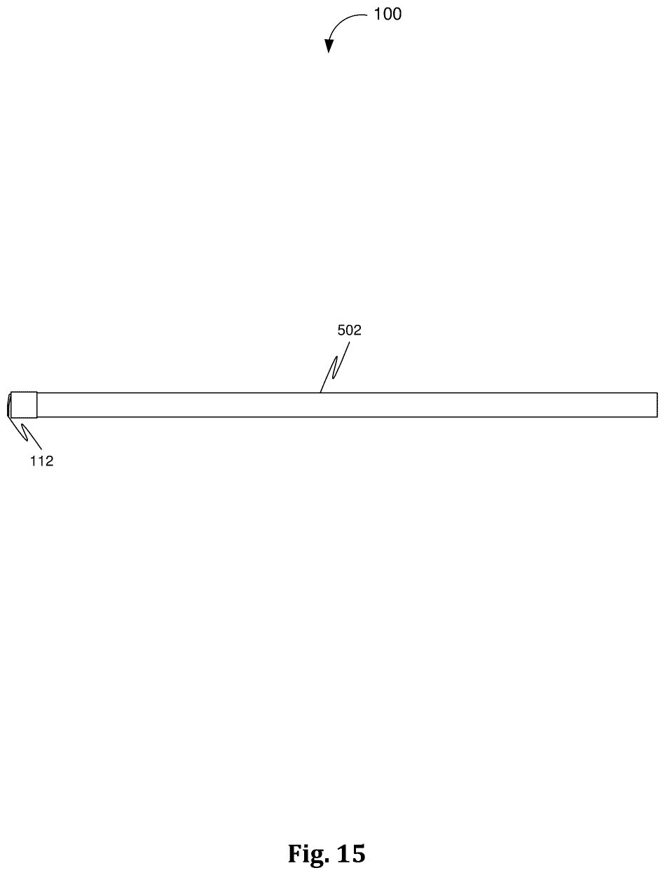

FIG. 15 illustrates a right side view of the apparatus 100 for fetal health monitoring including the delivery tube 502, in accordance with some embodiments. Further, the right side view of the apparatus 100 including the delivery tube 502 shows the electrode 112.

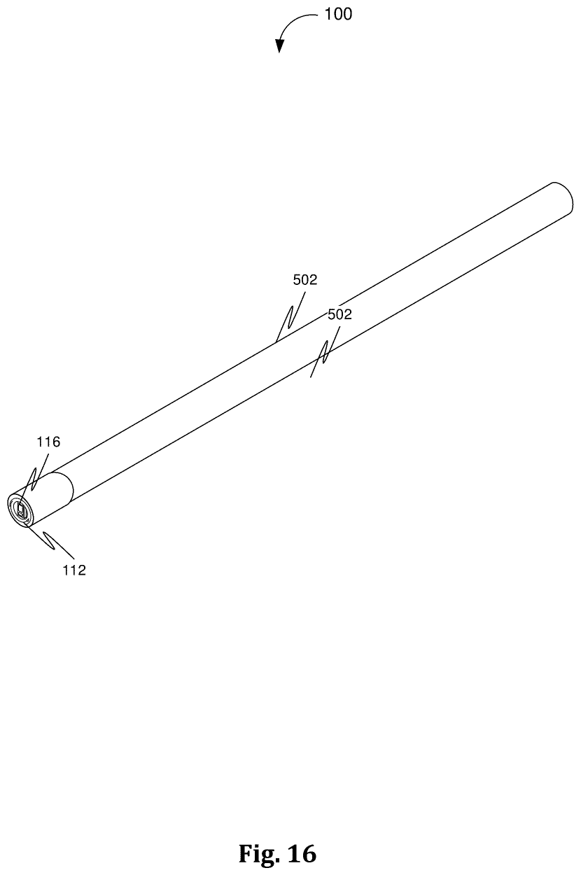

FIG. 16 illustrates a perspective view of the apparatus 100 for fetal health monitoring including the delivery tube 502, in accordance with some embodiments. Further, the perspective view of the apparatus 100 including the delivery tube 502 shows the one or more sensors 116 and the electrode 112.

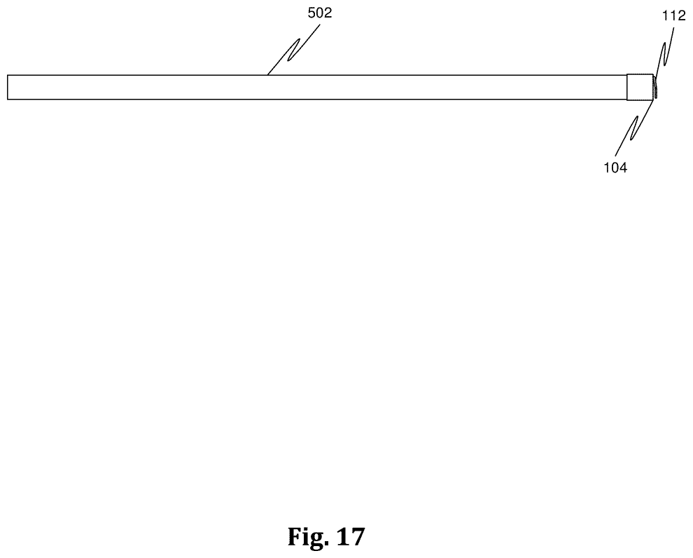

FIG. 17 illustrates a left side view of the apparatus 100 for fetal health monitoring including the delivery tube 502, in accordance with some embodiments. Further, the left side view of the apparatus 100 including the delivery tube 502 shows the electrode 112 extending from the first body end 104.

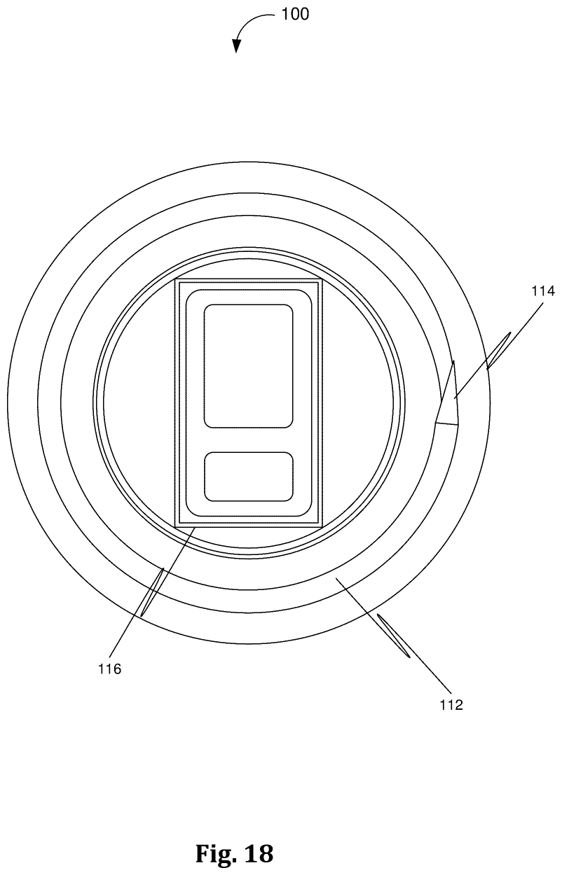

FIG. 18 illustrates a front view of the apparatus 100 for fetal health monitoring, in accordance with some embodiments. Further, the front view of the apparatus 100 including the delivery tube 502 shows the one or more sensors 116, the electrode 112, and the second electrode end 114.

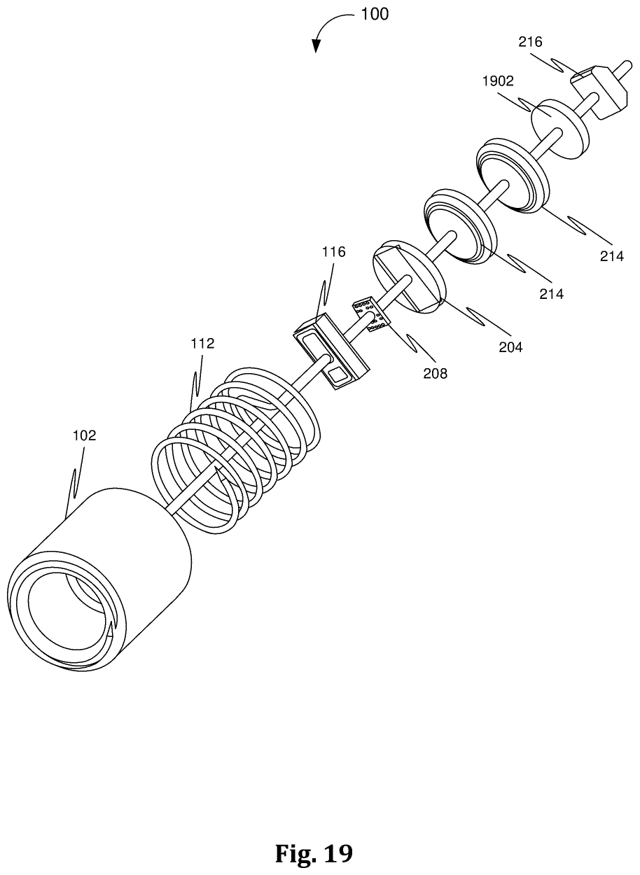

FIG. 19 illustrates an exploded view of the apparatus 100 for fetal health monitoring, in accordance with some embodiments. Further, the apparatus 100 may include an additional insulation disk 1902 between the magnetic reed switch 216 and the one or more batteries 214.

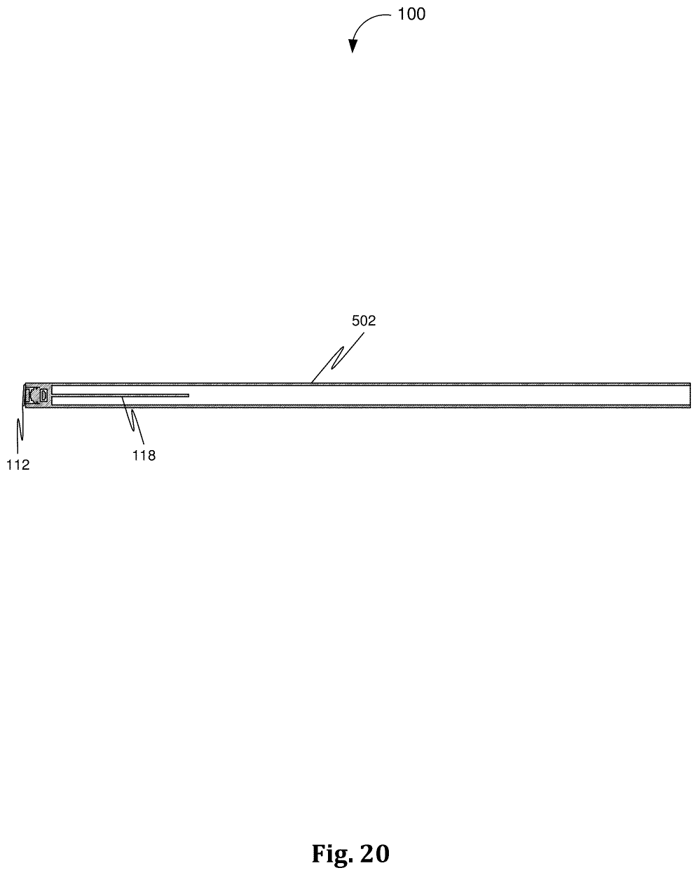

FIG. 20 illustrates a cross-sectional view of the apparatus 100 for fetal health monitoring including the delivery tube 502, in accordance with some embodiments. Further, the cross-sectional view of the apparatus 100 including the delivery tube 502 shows the electrode 112 and a Bluetooth antenna 118.

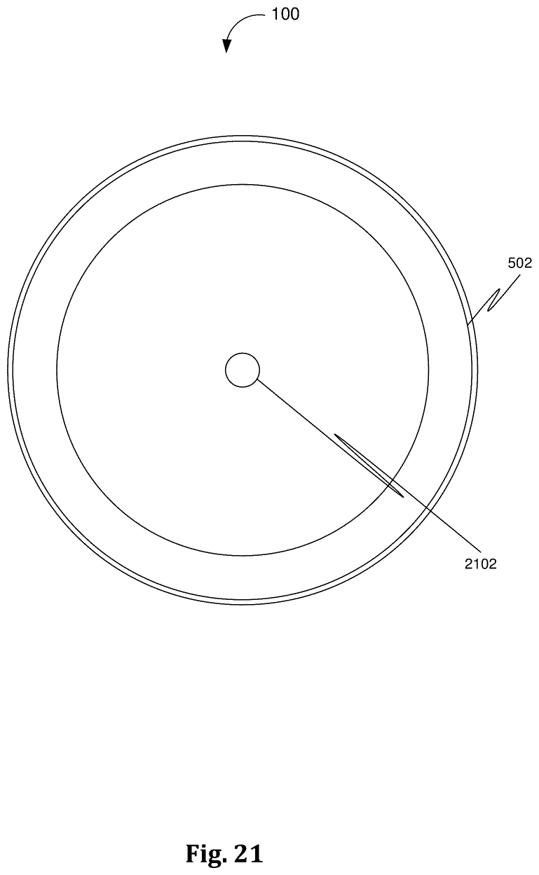

FIG. 21 illustrates a rear view of the apparatus 100 for fetal health monitoring including the delivery tube 502, in accordance with some embodiments. Further, the rear view of the apparatus 100 including the delivery tube 502 shows a tube opening 2102 of the delivery tube 502.

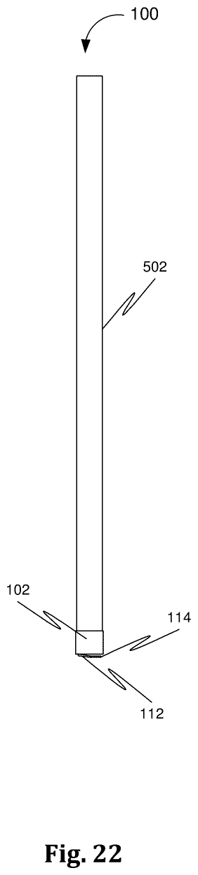

FIG. 22 illustrates a top view of the apparatus 100 for fetal health monitoring including the delivery tube 502, in accordance with some embodiments. Further, the top view of the apparatus 100 including the delivery tube 502 shows the electrode 112 and the second electrode end 114. Further, the second electrode end 114 may be helically extended from the body 102.

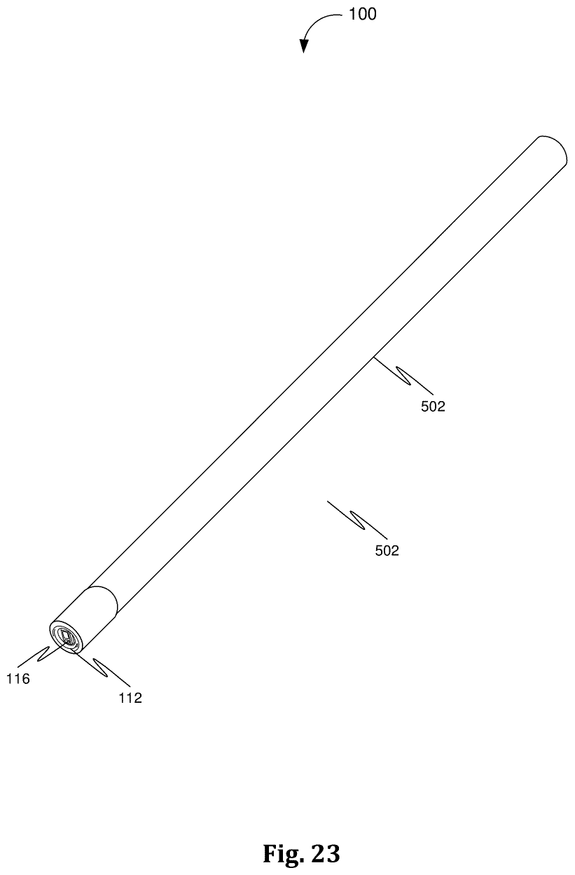

FIG. 23 illustrates an isometric view of the apparatus 100 for fetal health monitoring including the delivery tube 302, in accordance with some embodiments. Further, the isometric view of the apparatus 100 including the delivery tube 502 shows the electrode 112 and the one or more sensors 116.

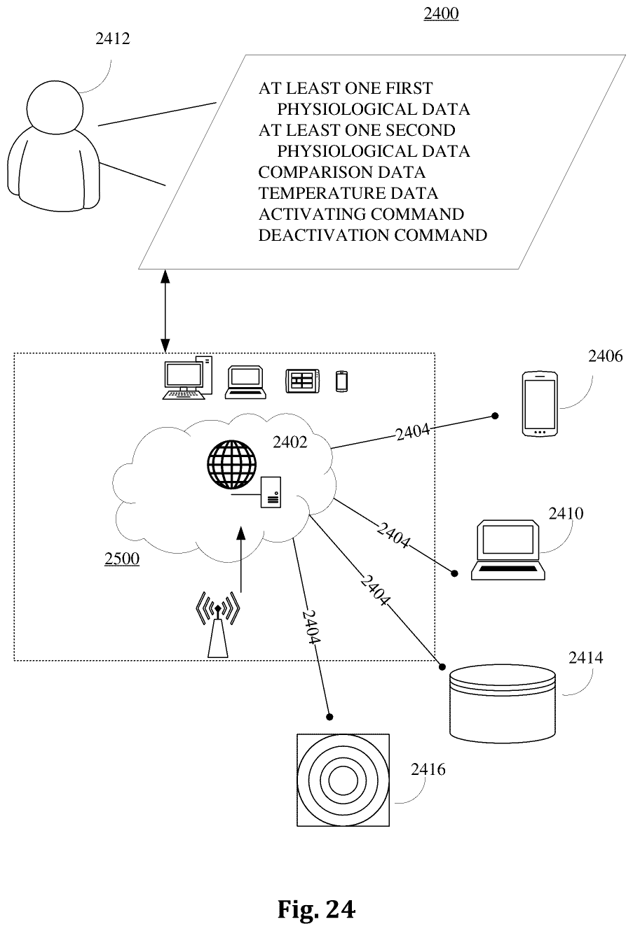

FIG. 24 is an illustration of an online platform 2400 consistent with various embodiments of the present disclosure. By way of non-limiting example, the online platform 2400 may be hosted on a centralized server 2402, such as, for example, a cloud computing service. The centralized server 2402 may communicate with other network entities, such as, for example, a mobile device 2406 (such as a smartphone, a laptop, a tablet computer etc.), other electronic devices 2410 (such as desktop computers, server computers etc.), databases 2414, and sensors 2416 over a communication network 2404, such as, but not limited to, the Internet. Further, users of the online platform 2400 may include relevant parties such as, but not limited to, end-users, administrators, service providers, service consumers and so on. Accordingly, in some instances, electronic devices operated by the one or more relevant parties may be in communication with the platform.

A user 2412, such as the one or more relevant parties, may access online platform 2400 through a web based software application or browser. The web based software application may be embodied as, for example, but not be limited to, a website, a web application, a desktop application, and a mobile application compatible with a computing device 2500.

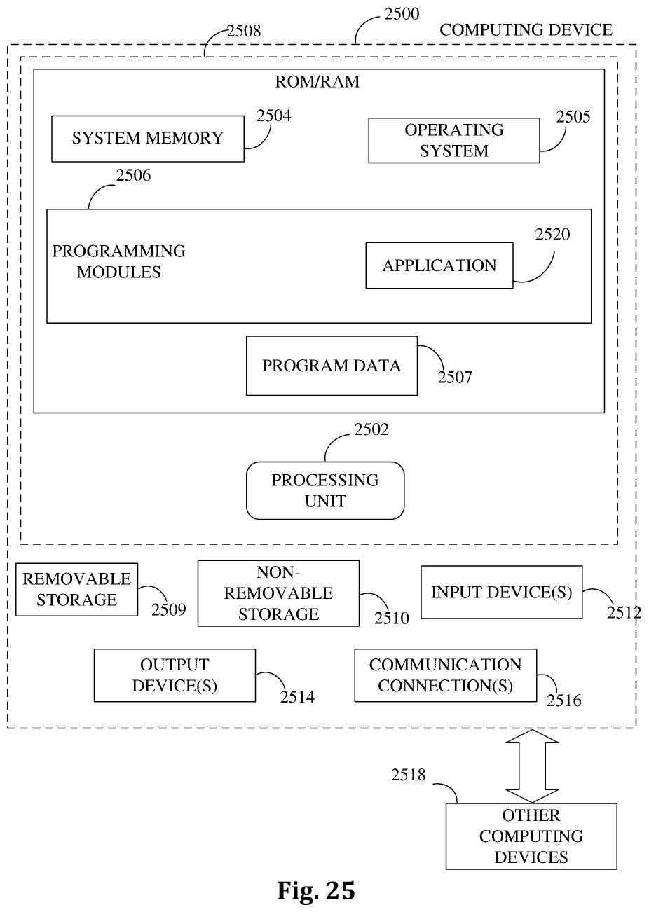

With reference to FIG. 25, a system consistent with an embodiment of the disclosure may include a computing device or cloud service, such as computing device 2500. In a basic configuration, computing device 2500 may include at least one processing unit 2502 and a system memory 2504. Depending on the configuration and type of computing device, system memory 2504 may comprise, but is not limited to, volatile (e.g. random-access memory (RAM)), non-volatile (e.g. read-only memory (ROM)), flash memory, or any combination. System memory 2504 may include operating system 2505, one or more programming modules 2506, and may include a program data 2507. Operating system 2505, for example, may be suitable for controlling computing device 2500's operation. In one embodiment, programming modules 2506 may include image-processing module, machine learning module. Furthermore, embodiments of the disclosure may be practiced in conjunction with a graphics library, other operating systems, or any other application program and is not limited to any particular application or system. This basic configuration is illustrated in FIG. 25 by those components within a dashed line 2508.

Computing device 2500 may have additional features or functionality. For example, computing device 2500 may also include additional data storage devices (removable and/or non-removable) such as, for example, magnetic disks, optical disks, or tape. Such additional storage is illustrated in FIG. 25 by a removable storage 2509 and a non-removable storage 2510. Computer storage media may include volatile and non-volatile, removable and non-removable media implemented in any method or technology for storage of information, such as computer-readable instructions, data structures, program modules, or other data. System memory 2504, removable storage 2509, and non-removable storage 2510 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 2500. Any such computer storage media may be part of device 2500. Computing device 2500 may also have input device(s) 2512 such as a keyboard, a mouse, a pen, a sound input device, a touch input device, a location sensor, a camera, a biometric sensor, etc. Output device(s) 2514 such as a display, speakers, a printer, etc. may also be included. The aforementioned devices are examples and others may be used.

Computing device 2500 may also contain a communication connection 2516 that may allow device 2500 to communicate with other computing devices 2518, such as over a network in a distributed computing environment, for example, an intranet or the Internet. Communication connection 2516 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 2504, including operating system 2505. While executing on processing unit 2502, programming modules 2506 (e.g., application 2520 such as a media player) may perform processes including, for example, one or more stages of methods, algorithms, systems, applications, servers, databases as described above. The aforementioned process is an example, and processing unit 2502 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

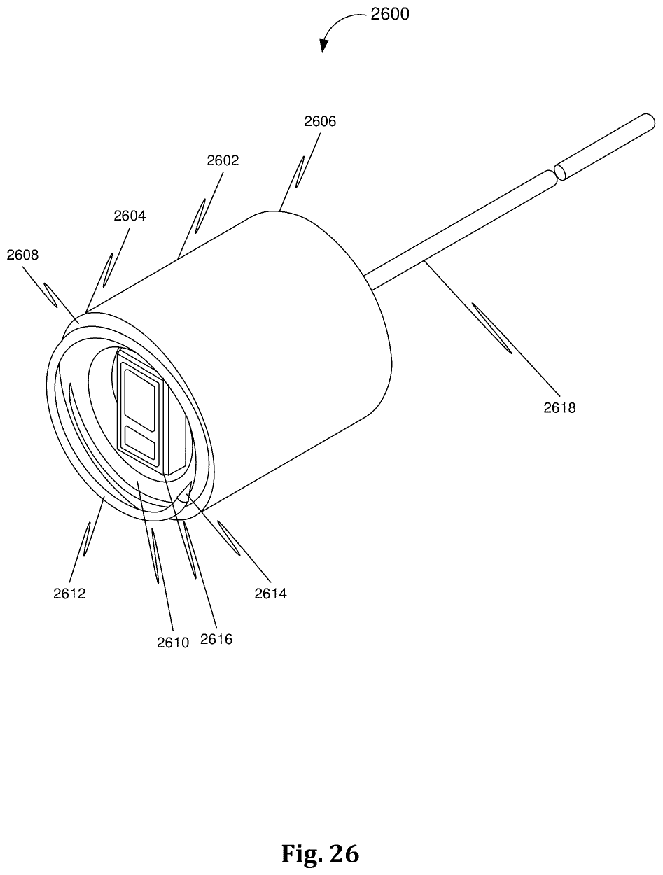

FIG. 26 illustrates an apparatus 2600 for fetal health monitoring, in accordance with some embodiments. Further, the apparatus 2600 may include a body 2602 extending between a first body end 2604 and a second body end 2606. Further, the body 2602 includes a body cavity 2610. Further, the first body end 2604 defines a body opening 2608 leading into the body cavity 2610. Further, the body 2602 may be configured to be inserted into one or more user body cavities of one or more users. Further, the apparatus 2600 may include an electrode 2612 extending between a first electrode end and a second electrode end 2614. Further, the electrode 2612 may be helically extending from the body 2602. Further, the first electrode end may be attached to the body 2602, and the second electrode end 2614 may be free. Further, the second electrode end 2614 may be configured to be inserted into one or more body parts of a fetus for securing the apparatus 2600 to the one or more body parts based on one or more action receivables on the body 2602. Further, the electrode 2612 may be configured for detecting one or more electrical signals associated with the one or more body parts of the fetus based on the inserting of the second electrode end 2614 into the one or more body parts of the fetus. Further, the one or more electrical signals may be associated with one or more first physiological parameters of the fetus. Further, the electrode 2612 further includes an electrode cavity extending from the second electrode end 2614. Further, the second electrode end 2614 defines an electrode opening leading into the electrode cavity. Further, the electrode 2612 further includes a sample collecting mechanism coupled with the electrode cavity. Further, the sample collecting mechanism may be configured to be activated. Further, the sample collecting mechanism may be configured for collecting one or more samples associated with the fetus based on the inserting of the body 102 into the one or more user body cavities of the one or more users after the activating of the sample collecting mechanism. Further, the electrode 2612 may be configured for storing the one or more samples in the electrode cavity based on the collecting of the one or more samples. Further, the apparatus 2600 may include one or more sensors 2616 disposed in the body cavity 2610. Further, the one or more sensors 2616 may be configured for detecting one or more second physiological parameters associated with the fetus. Further, the apparatus 2600 may include a processing device disposed within the body 2602. Further, the processing device may be communicatively coupled with the electrode 2612 and the one or more sensors 2616. Further, the processing device may be configured for generating one or more first physiological data based on the detecting of the one or more electrical signals. Further, the processing device may be configured for generating one or more second physiological data based on the detecting of the one or more second physiological parameters. Further, the apparatus 2600 may include a communication device disposed within the body 2602. Further, the communication device may be communicatively coupled with the processing device. Further, the communication device may be configured for transmitting the one or more first physiological data and the one or more second physiological data to one or more external devices.

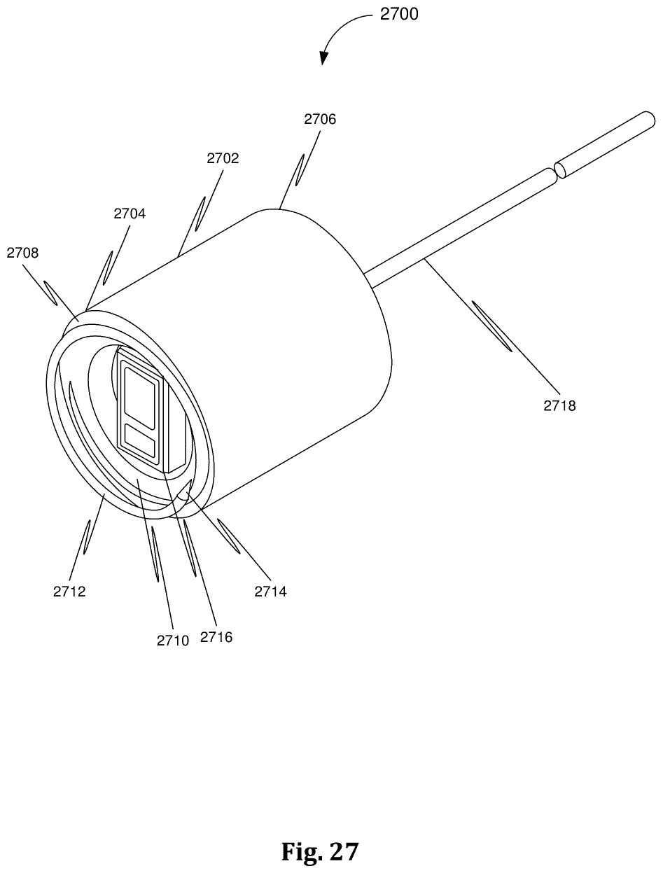

FIG. 27 illustrates an apparatus 2700 for fetal health monitoring, in accordance with some embodiments. Further, the apparatus 2700 may include a body 2702 extending between a first body end 2704 and a second body end 2706. Further, the body 2702 includes a body cavity 2710. Further, the first body end 2704 defines a body opening 2708 leading into the body cavity 2710. Further, the body 2702 may be configured to be inserted into one or more user body cavities of one or more users. Further, the apparatus 2700 may include an electrode 2712 extending between a first electrode end and a second electrode end 2714. Further, the electrode 2712 may be helically extending from the body 2702. Further, the first electrode end may be attached to the body 2702, and the second electrode end 2714 may be free. Further, the second electrode end 2714 may be configured to be inserted into one or more body parts of a fetus for securing the apparatus 2700 to the one or more body parts based on one or more action receivables on the body 2702. Further, the electrode 2712 may be configured for detecting one or more electrical signals associated with the one or more body parts of the fetus based on the inserting of the second electrode end 2714 into the one or more body parts of the fetus. Further, the one or more electrical signals may be associated with one or more first physiological parameters of the fetus. Further, the apparatus 2700 may include one or more sensors 2716 disposed in the body cavity 2710. Further, the one or more sensors 2716 may be configured for detecting one or more second physiological parameters associated with the fetus. Further, the apparatus 2700 may include a processing device disposed within the body 2702. Further, the processing device may be communicatively coupled with the electrode 2712 and the one or more sensors 2716. Further, the processing device may be configured for generating one or more first physiological data based on the detecting of the one or more electrical signals. Further, the processing device may be configured for generating one or more second physiological data based on the detecting of the one or more second physiological parameters. Further, the apparatus 2700 may include a communication device disposed within the body 2702. Further, the communication device may be communicatively coupled with the processing device. Further, the communication device may be configured for transmitting the one or more first physiological data and the one or more second physiological data to one or more external devices. Further, the apparatus 2700 may include a delivery tube extending between a first tube end and a second tube end. Further, the delivery tube includes a tube cavity extending between the first tube end and the second tube end. Further, the body 2702 may be disposed in the tube cavity at the first tube end. Further, the first tube end may be inserted in the one or more user body cavities. Further, the first tube end may be configured to be positioned in one or more positions in the one or more user body cavities in relation to the fetus after the inserting of the first tube end. Further, the inserting of the second electrode end 2714 into the one or more body parts of the fetus may be based on the positioning of the first tube end in the one or more positions. Further, the one or more action receivables on the body 2702 may be applied using the delivery tube.

The present disclosure provides a continuous intrapartum fetal monitoring device. Further, the continuous intrapartum fetal monitoring device may include a sensor body which may be configured for placement within a vaginal canal of a laboring patient. Further, the sensor body defines a recessed cavity oriented toward a fetal scalp. Further, the continuous intrapartum fetal monitoring device may include a fixation structure coupled to the sensor body. Further, the fixation structure includes a helical electrode which may be configured to engage a superficial layer of a fetal scalp tissue to secure the continuous intrapartum fetal monitoring device in position during continuous monitoring. Further, the continuous intrapartum fetal monitoring device may include one or more optical emitters disposed within the recessed cavity. Further, the one or more optical emitters may be configured to emit a light at two or more wavelengths. Further, the two or more wavelengths include approximately 660 nanometers and approximately 940 nanometers. Further, the continuous intrapartum fetal monitoring device may include one or more optical detectors positioned to receive a light reflected from the fetal scalp tissue and generate photoplethysmographic signals. Further, the continuous intrapartum fetal monitoring device may include one or more electrical electrodes positioned on or adjacent to the fixation structure. Further, the one or more electrical electrodes may be configured to receive fetal electrocardiographic signals. Further, the continuous intrapartum fetal monitoring device may include a processor disposed within the sensor body. Further, the processor may be configured for calculating one or more of a fetal heart rate and an arterial oxygen saturation (SpO₂) from the photoplethysmographic signals, and extracting a fetal heart rate from the fetal electrocardiographic signals. Further, the continuous intrapartum fetal monitoring device may include a wireless communication module within the sensor body which may be configured to transmit a calculated SpO₂ and fetal heart rate data to an external monitor for display in real time.

In some embodiments, the recessed cavity further includes one or more of an opaque barrier, an optical baffle, and a window which may be configured to minimize ambient light interference from a maternal environment.

In some embodiments, the fixation structure further includes a suction or negative-pressure channel which may be configured to draw the fetal scalp tissue into contact with the recessed cavity to improve an optical coupling.

In some embodiments, the one or more optical emitters and the one or more detectors may be mounted on a flexible printed circuit board having a vertical portion extending into the recessed cavity and a horizontal portion contained within the sensor body.

In some embodiments, the processor may be configured to filter photoplethysmographic data of the photoplethysmographic signals to reject signals corresponding to maternal pulse interference.

In some embodiments, the processor may be configured to provide continuous SpO₂ trend data for at least thirty minutes of monitoring.

In some embodiments, the wireless communication module uses a Bluetooth Low Energy (BLE) protocol to transmit the calculated SpO2 and fetal heart rate data to a paired monitoring system.

In some embodiments, the one or more electrical electrodes provide both fixation and signal acquisition functions.

In some embodiments, the recessed cavity includes a transparent biocompatible optical window formed of a hydrophobic material.

In some embodiments, the processor may include an artifact rejection algorithm which may be configured to filter motion and uterine contraction-induced noise.

In some embodiments, the processor may be configured to synchronize transmitted data with external cardiotocography (CTG) recordings.

In some embodiments, the sensor body includes a breakaway or release feature enabling detachment of a delivery tube after insertion.

In some embodiments, the processor and the wireless communication module may be powered by an integrated micro-battery or inductive power source.

In some embodiments, the system may be configured for continuous use during the active phase of labor and provide real-time feedback to guide intrapartum clinical decisions.

The present disclosure provides a fetal monitoring system including the continuous intrapartum fetal monitoring device and an external monitor. Further, the fetal monitoring system may be configured to receive transmitted SpO₂ data and ECG data. Further, the fetal monitoring system may be configured to compute and display time-synchronized fetal SpO₂ and heart rate trends. Further, the fetal monitoring system be configured to compare said trends to maternal vital signs and uterine contraction data. Further, the fetal monitoring system may be configured to generate an alert when fetal oxygenation falls below a threshold or diverges from expected CTG patterns.

The present disclosure provides a method of continuously monitoring fetal condition during labor. Further, the method of continuously monitoring fetal condition during labor may include inserting a fetal sensor device into a vaginal canal of a laboring patient such that a helical fixation element engages a fetal scalp. Further, the method of continuously monitoring fetal condition during labor may include emitting a light from one or more optical emitters at two or more wavelengths including approximately 660 nanometers and approximately 940 nanometers toward a fetal scalp tissue. Further, the method of continuously monitoring fetal condition during labor may include detecting reflected light with one or more optical detectors to generate photoplethysmographic signals. Further, the method of continuously monitoring fetal condition during labor may include calculating arterial oxygen saturation (SpO₂) values from the photoplethysmographic signals. Further, the method of continuously monitoring fetal condition during labor may include simultaneously acquiring fetal electrocardiographic (ECG)signals through one or more electrodes associated with the helical fixation element. Further, the method of continuously monitoring fetal condition during labor may include calculating fetal heart rate from the ECG signals. Further, the method of continuously monitoring fetal condition during labor may include transmitting a calculated SpO₂ and fetal heart rate data to an external monitor for real-time display and analysis.

In some embodiments, the external monitor further displays synchronized uterine contraction data and cardiotocography (CTG) classifications alongside a fetal SpO₂ and heart rate trends.

In some embodiments, the method may include filtering maternal pulse interference and motion artifacts from the photoplethysmographic signals prior to calculating SpO₂.

In some embodiments, a step of calculating SpO₂ includes determining a ratio-of-ratios based on a red and infrared wavelength component.

In some embodiments, the device transmits data via a Bluetooth Low Energy (BLE) communication protocol.

In some embodiments, the optical detection occurs continuously for a monitoring period of at least thirty minutes during active labor.

In some embodiments, the external monitor compares a fetal SpO₂ value to predetermined thresholds and generates an alert when oxygenation levels fall below a critical value.

In some embodiments, the transmitted data may be stored in a patient monitoring database for later clinical review or machine-learning-based analysis of fetal well-being.

In some embodiments, the device may be powered by an internal rechargeable battery or an inductive power system to permit untethered operation during labor.

In some embodiments, the continuous intrapartum fetal monitoring device may further include one or more temperature sensors disposed adjacent to the one or more optical emitters. Further, the one or more temperature sensors may be configured to measure fetal scalp surface temperature.

In some embodiments, the processor may be configured to correlate a temperature data with the SpO₂ and fetal heart rate data to assess fetal perfusion status.

In some embodiments, the sensor array further includes one or more impedance or conductivity sensors which may be configured to detect fluid presence or tissue contact quality.

In some embodiments, the processor may be configured to compute a composite fetal well-being index based on SpO₂, heart rate, and temperature values.

In some embodiments, the method may further include a step of measuring fetal scalp temperature via an integrated temperature sensor.

In some embodiments, the method may further include correlating the temperature data with SpO₂ and ECG data to evaluate fetal tissue perfusion.

Although the invention has been explained in relation to its preferred embodiment, it is to be understood that many other possible modifications and variations can be made without departing from the spirit and scope of the invention as hereinafter claimed.

What is claimed is:

1. An apparatus for fetal health monitoring, the apparatus comprising:
a body extending between a first body end and a second body end, wherein the body comprises a body cavity, wherein the first body end defines a body opening leading into the body cavity, wherein the body is configured to be inserted into at least one user body cavity of at least one user;
an electrode extending between a first electrode end and a second electrode end, wherein the electrode is helically extending from the body, wherein the first electrode end is attached to the body, and the second electrode end is free, wherein the second electrode end is configured to be inserted into at least one body part of a fetus for securing the apparatus to the at least one body part based on at least one action receivable on the body, wherein the electrode is configured for detecting at least one electrical signal associated with the at least one body part of the fetus based on the inserting of the second electrode end into the at least one body part of the fetus, wherein the at least one electrical signal is associated with at least one first physiological parameter of the fetus;
at least one sensor disposed in the body cavity, wherein the at least one sensor is configured for detecting at least one second physiological parameter associated with the fetus;
a processing device disposed within the body, wherein the processing device is communicatively coupled with the electrode and the at least one sensor, wherein the processing device is configured for:
generating at least one first physiological data based on the detecting of the at least one electrical signal; and
generating at least one second physiological data based on the detecting of the at least one second physiological parameter; and
a communication device disposed within the body, wherein the communication device is communicatively coupled with the processing device, wherein the communication device is configured for transmitting the at least one first physiological data and the at least one second physiological data to at least one external device.

2. The apparatus of claim 1, wherein the electrode further comprises an electrode cavity extending from the second electrode end, wherein the second electrode end defines an electrode opening leading into the electrode cavity, wherein the electrode further comprises a sample collecting mechanism coupled with the electrode cavity, wherein the sample collecting mechanism is configured to be activated, wherein the sample collecting mechanism is configured for collecting at least one sample associated with the fetus based on the inserting of the body into the at least one user body cavity of the at least one user after the activating of the sample collecting mechanism, wherein the electrode is configured for storing the at least one sample in the electrode cavity based on the collecting of the at least one sample.

3. The apparatus of claim 2, wherein the electrode further comprises a lid coupled to a portion of the electrode, wherein the lid is configured to be transitioned between a closed position and an open position, wherein the lid covers the electrode opening in the closed position, and the lid uncovers the electrode opening in the open position.

4. The apparatus of claim 3, wherein the electrode further comprises a lid transitioning mechanism, wherein the lid transitioning mechanism is configured for transitioning between a first state and a second state, wherein the lid transitions to the closed position is based on the transitioning of the lid transitioning mechanism from the first state to the second state, wherein the lid transitions to the open position is based on the transitioning of the lid transitioning mechanism from the second state to the first state, wherein the collecting of the at least one sample is further based on the transitioning of the lid transitioning mechanism from the second state to the first state.

5. The apparatus of claim 2, wherein the communication device is configured for receiving an activating command for the activating of the sample collecting mechanism, wherein the processing device is further configured for activating the sample collecting mechanism based on the activating command.

6. The apparatus of claim 2, wherein the sample collecting mechanism is configured to be deactivated, wherein the deactivating of the sample collecting mechanism halts the collecting of the at least one sample.

7. The apparatus of claim 6, wherein the communication device is configured for receiving a deactivation command for the deactivating of the sample collecting mechanism, wherein the processing device is further configured for deactivating the sample collecting mechanism based on the deactivating command.

8. The apparatus of claim 4, wherein the lid transitioning mechanism is operatively coupled with the sample collecting mechanism, wherein the transitioning of the lid transitioning mechanism from the second state to the first state is based on the activating of the sample collecting mechanism.

9. The apparatus of claim 1 further comprising a delivery tube extending between a first tube end and a second tube end, wherein the delivery tube comprises a tube cavity extending between the first tube end and the second tube end, wherein the body is disposed in the tube cavity at the first tube end, wherein the first tube end is inserted in the at least one user body cavity, wherein the first tube end is configured to be positioned in at least one position in the at least one user body cavity in relation to the fetus after the inserting of the first tube end, wherein the inserting of the second electrode end into the at least one body part of the fetus is based on the positioning of the first tube end in the at least one position, wherein the at least one action receivable on the body is applied using the delivery tube.

10. The apparatus of claim 9, wherein the delivery tube is detached from the body after the inserting of the second electrode end into the at least one body part of the fetus, wherein the detaching of the delivery tube is based on at least one second action applied on the delivery tube.

11. The apparatus of claim 1 further comprising a printed circuit board (PCB) disposed in the body cavity, wherein the PCB comprises each of the processing device and the communication device, wherein each of the at least one sensor and the first electrode end of the electrode is mounted on the PCB.

12. The apparatus of claim 1, wherein the at least one sensor disposed in the body cavity is recessed inward in relation to the body opening, wherein the apparatus further comprises a suction mechanism disposed in the body cavity, wherein the suction mechanism is configured for extending a portion of a skin of the at least one body part into the body cavity based on the securing of the apparatus to the at least one body part, wherein the extending of the portion of the skin into the cavity for establishing a direct contact of the portion of the skin with at least one sensing element of the at least one sensor, wherein the detecting of the at least one second physiological parameter is further based on the establishing of the direct contact of the portion of the skin with the at least one sensing element.

13. The apparatus of claim 1, wherein the body comprises a body portion defining the body cavity, wherein the body portion is configured to be deformed, wherein the at least one sensor disposed in the body cavity is recessed inward in relation to the body opening, wherein the deforming of the body portion after the securing of the apparatus to the at least one body part makes at least one sensing element of the at least one sensor to establish a direct contact with a portion of a skin of the at least one body part, wherein the detecting of the at least one second physiological parameter is further based on the establishing of the direct contact of the portion of the skin with the at least one sensing element.

14. The apparatus of claim 1, wherein the body comprises an inner base surface and an inner side surface defining the body cavity, wherein the inner side surface extends from the inner base surface towards the body opening, wherein the at least one sensor is attached to the inner base surface defining the body cavity using an attachment mechanism, wherein the securing of the apparatus to the at least one body part results in a gap between a portion of a skin of the at least one body part and the inner base surface, wherein the attachment mechanism is configured for retractably extending the at least one sensor in the gap for transitioning the at least one sensor between an extended position and a retracted position, wherein the at least one sensor is recessed inward in relation to the body opening in the retracted position, wherein at least one sensing element of the at least one sensor is in a direct contact with the portion of the skin in the extended position, wherein the detecting of the at least one second physiological parameter is further based on establishing of the direct contact of the portion of the skin with the at least one sensing element.

15. The apparatus of claim 1, wherein the at least one sensor disposed in the body cavity is recessed inward in relation the body opening, wherein the securing of the apparatus to the at least one body part results in a gap between a portion of a skin of the at least one body part and at least one sensing element of the at least one sensor, wherein the detecting of the at least one second physiological parameter comprises performing at least one sensing operation by the at least one sensor using the at least one sensing element without a direct contact between the at least one sensing element and the portion of the skin of the at least one body part.

16. The apparatus of claim 11 further comprising at least one battery disposed within the body, wherein the at least one battery is electrically coupled with the PCB, wherein each of the processing device, the communication device, and the at least one sensor is electrically powered.

17. The apparatus of claim 1, wherein the body comprises an optical shield disposed on the body, wherein the optical shield covers the body opening of the body, wherein the at least one sensor is configured for performing at least one sensing operation through the optical shield, wherein the detecting of the at least one second physiological parameter associated with the fetus is further based on the performing of the at least one sensing operation.

18. The apparatus of claim 11 further comprising a temperature sensor disposed in the body cavity, wherein the temperature sensor is placed adjacent to the at least one sensor in the body cavity, wherein the temperature sensor is configured for detecting a temperature of the fetus, wherein the temperature sensor is mounted on the PCB, wherein the processing device is communicatively coupled with the temperature sensor, wherein the processing device is further configured for generating a temperature data based on the detecting of the temperature of the fetus, wherein the communication device is further configured for transmitting the temperature data to the at least one external device.

19. An apparatus for fetal health monitoring, the apparatus comprising:
a body extending between a first body end and a second body end, wherein the body comprises a body cavity, wherein the first body end defines a body opening leading into the body cavity, wherein the body is configured to be inserted into at least one user body cavity of at least one user;
an electrode extending between a first electrode end and a second electrode end, wherein the electrode is helically extending from the body, wherein the first electrode end is attached to the body, and the second electrode end is free, wherein the second electrode end is configured to be inserted into at least one body part of a fetus for securing the apparatus to the at least one body part based on at least one action receivable on the body, wherein the electrode is configured for detecting at least one electrical signal associated with the at least one body part of the fetus based on the inserting of the second electrode end into the at least one body part of the fetus, wherein the at least one electrical signal is associated with at least one first physiological parameter of the fetus, wherein the electrode further comprises an electrode cavity extending from the second electrode end, wherein the second electrode end defines an electrode opening leading into the electrode cavity, wherein the electrode further comprises a sample collecting mechanism coupled with the electrode cavity, wherein the sample collecting mechanism is configured to be activated, wherein the sample collecting mechanism is configured for collecting at least one sample associated with the fetus based on the inserting of the body into the at least one user body cavity of the at least one user after the activating of the sample collecting mechanism, wherein the electrode is configured for storing the at least one sample in the electrode cavity based on the collecting of the at least one sample;
at least one sensor disposed in the body cavity, wherein the at least one sensor is configured for detecting at least one second physiological parameter associated with the fetus;
a processing device disposed within the body, wherein the processing device is communicatively coupled with the electrode and the at least one sensor, wherein the processing device is configured for:
generating at least one first physiological data based on the detecting of the at least one electrical signal; and
generating at least one second physiological data based on the detecting of the at least one second physiological parameter; and
a communication device disposed within the body, wherein the communication device is communicatively coupled with the processing device, wherein the communication device is configured for transmitting the at least one first physiological data and the at least one second physiological data to at least one external device.

20. An apparatus for fetal health monitoring, the apparatus comprising:
a body extending between a first body end and a second body end, wherein the body comprises a body cavity, wherein the first body end defines a body opening leading into the body cavity, wherein the body is configured to be inserted into at least one user body cavity of at least one user;
an electrode extending between a first electrode end and a second electrode end, wherein the electrode is helically extending from the body, wherein the first electrode end is attached to the body, and the second electrode end is free, wherein the second electrode end is configured to be inserted into at least one body part of a fetus for securing the apparatus to the at least one body part based on at least one action receivable on the body, wherein the electrode is configured for detecting at least one electrical signal associated with the at least one body part of the fetus based on the inserting of the second electrode end into the at least one body part of the fetus, wherein the at least one electrical signal is associated with at least one first physiological parameter of the fetus;
at least one sensor disposed in the body cavity, wherein the at least one sensor is configured for detecting at least one second physiological parameter associated with the fetus;
a processing device disposed within the body, wherein the processing device is communicatively coupled with the electrode and the at least one sensor, wherein the processing device is configured for:
generating at least one first physiological data based on the detecting of the at least one electrical signal; and
generating at least one second physiological data based on the detecting of the at least one second physiological parameter;
a communication device disposed within the body, wherein the communication device is communicatively coupled with the processing device, wherein the communication device is configured for transmitting the at least one first physiological data and the at least one second physiological data to at least one external device; and
a delivery tube extending between a first tube end and a second tube end, wherein the delivery tube comprises a tube cavity extending between the first tube end and the second tube end, wherein the body is disposed in the tube cavity at the first tube end, wherein the first tube end is inserted in the at least one user body cavity, wherein the first tube end is configured to be positioned in at least one position in the at least one user body cavity in relation to the fetus after the inserting of the first tube end, wherein the inserting of the second electrode end into the at least one body part of the fetus is based on the positioning of the first tube end in the at least one position, wherein the at least one action receivable on the body is applied using the delivery tube.
